(19) United States
(12) Patent Application Publication
(10) Pub. No.: US 2015/0030635 A1
(43) Pub. Date: Jan. 29, 2015

(54) Activated Immunostimulatory Cell Composition and Uses Thereof

(71) Applicant: Macrocure, Ltd., Kiryat Matalon (IL)

(72) Inventors: Irene Ginis, Beit Shemesh (IL); Alan Smith, Columbia, MD (US); Adi Zuloff-Shani, Herzliyya (IL); Marina Bubis, Rehovot (IL); Mitchell Shirvan, Herzliyya (IL)

(21) Appl. No.: 14/384,872

(22) PCT Filed: Mar. 13, 2013
(86) PCT No.: PCT/IB2013/000848

§ 371 Date: Sep. 12, 2014

Related U.S. Application Data
(60) Provisional application No. 61/611,202, filed on Mar. 15, 2012.

(51) Int. Cl. C12N 5/0784; A61K 35/14; A61K 39/00

(52) U.S. Cl. CPC C12N 5/0639; A61K 39/0011; A61K 35/14; A61K 2039/515
USPC 424/277.1; 424/93.71; 435/375

(57) ABSTRACT

Methods of making activated immunostimulatory cell compositions, activated immunostimulatory cell compositions, and methods of using those compositions to stimulate therapeutic immune responses to tumors are described.

### CROSS-REFERENCE TO RELATED APPLICATIONS

This application claims the benefit of U.S. Provisional Application No. 61/611,202, filed Mar. 15, 2012, which is incorporated herein by reference.

### DESCRIPTION OF THE INVENTION

### Field and Background

The present invention relates to activated immunostimulatory cell compositions, methods of preparing those compositions, and to uses of the compositions to treat conditions that may benefit from immunostimulation, such as cancer.

Many tumors possess tumor-specific and tumor-associated antigens. Tumor specific antigens (TSA) are uniquely expressed on the tumor and not present on the normal cells while tumor-associated antigens (TAA) are expressed at higher levels on tumors than on normal cells (Wang, R F 2002). Tumor antigens are cleaved within tumor cells into small peptides that are presented to immune cells as a complex with class I Major Histocompatibility molecules (MHC class I) also called Human Leukocyte Antigens (HLA class I, e.g. HLA A, B, or C). The MHC class I-tumor peptide complex is recognized by the T Cell Receptor (TCR) complex of cytotoxic T lymphocytes (CTLs), which are CD8⁺ T cells. The interaction of TCR with MHC I-tumor peptide complex causes release of substances from CTLs that destroy tumor cells (perforin, granzymes, and granulysin) (Mami-Chouaib F, 2002). CTLs also send death or apoptotic signals (through FAS receptor) to tumor cells. (Wu J Y 2006.)

The initial CTL response is short-lived and requires amplification support from CD4⁺ T helper (Th) lymphocytes to continue. Differentiation and proliferation of Th cells occurs through their interaction with professional Antigen Presenting Cells (APCs), such as macrophages or interstitial dendritic cells (DCs) (Wang R F. 2002). APCs engulf tumor antigens or apoptotic tumor cells, process these antigens by cleaving them into small peptides, then present the peptides to naïve T cells. The process is similar to CTL stimulation by MHC class I+peptide, but CD4⁺ T cells recognize peptides presented in the context of MHC class II (e.g. HLA-DR). Upon recognition of MHC class II-peptide complexes by TCR, T cells acquire Th functions and produce IL-2, a cytokine that in turn amplifies the CTL response and induces development of T memory cells for sustained immunity (Keene J A, 1982; Fujiwara, H, 1986).

T cell stimulation is thought to occur in at least two steps. (Marelli-Berg F M, 2007; Chambers C A 2001.) In the first step (signal 1), MHC/peptide complexes interact with the TCR. This step is not sufficient to fully stimulate the T cells. Instead, a second interaction between one of the APC-expressed co-stimulatory molecules (such as CD86, CD83, CD80 and CD40) and a corresponding ligand on T cells (signal 2) is required. T cells that receive signal 1 in the absence of signal 2 are unable to acquire helper functions (Chappert & Schwartz, 2010).

The interaction of naïve T helper cells with APCs such as DCs polarizes the Th cells into Th1 and Th2 subsets, which differ by the patterns of cytokines that they produce. Th1 cells produce cytokines such as interferons and IL-2 that activate proliferation of CTLs and cause tumor rejection. Th2 cells produce the cytokines IL-4, IL-6, IL-10 and IL-13. Increased levels of Th2 cytokines are found in sera of cancer patients with poor clinical prognosis.

Like Th cells, monocytes/macrophages phenotypes polarize in a process that is dependent on the balance between Th1/Th2 cytokines. The nomenclature for these polarized cells mirrors the Th1 and Th2 nomenclature and, like Th1 and Th2 T cells, M1 and M2 macrophages produce different patterns of cytokines. M1 macrophages predominantly produce IL-12, IL-23, TNFα, IL-1, IL-6; whereas M2 macrophages produce high levels of IL-10 and IL-13 (Cassetta L, 2011). M1 macrophages also express high levels of HLA-DR, while M2 macrophages express high levels of CD163 antigen. Fully polarized M1 and M2 macrophages are the extremes of a continuum of functional states. Notably, macrophages that infiltrate tumor tissues are driven by the tumor environment to acquire a polarized M2 phenotype which plays a key role in subversion of adaptive immunity and inflammatory circuits to promote tumor growth and progression. In contrast, M1 macrophages elicit an anti-tumor effect.

Another APC that is thought to also differentiate from peripheral blood monocytes is the myeloid dendritic cell (DC). Mature, activated DCs are extremely potent APCs. DC maturation is associated with up-regulation of MHC molecules, co-stimulatory molecules (CD86, CD83 CD80, CD40) (Tuyaerts S, 2007), adhesion molecules, such as CD11b, CD11c, and CD54, and the chemokine receptor CCR7 (Alvarez D, 2008). The latter enables the DC to migrate from the peripheral tissue through the vessel walls to the T cell areas (Forster R, 2008). DC maturation not only ensures expression of molecules relevant for T cell stimulation, it also permits DC to reach the appropriate anatomical compartments in secondary lymphoid organs so that they can present antigens to naïve T cells. Cognate signals from T cells further activate DC (Cavanagh L L, 2002).

Mature myeloid DCs are characterized by their ability to make IL-12 (Mariotti S, 2008). Because IL-12 promotes Th1 polarization, DCs that produce IL-12 are used in cancer vaccine development (Trinchieri G: 2003).

In addition to stimulating the adaptive, antigen-specific immune responses described above, DCs also play a role in stimulating innate (MHC-unrestricted) immunity, including stimulating several antitumor cell types. These cells include classical natural killer cells (NK cells), which do not express TCR (CD3−/CD56+ cells) and cytokine-induced killer T cells (CD3+/CD56+). NK cells can directly induce tumor cell apoptosis via the perforin-granzyme pathway or by expressing death-receptor ligands such as Fas ligand (Bryceson Y T, 2011). IL-12-producing DCs can induce proliferation of NK cells (Walzer T, 2005). NK cells also cells release cytokines that promote differentiation of DCs (Ferlazzo G, 2009).

Thus, mature DC are an important cell type in generating both effective adaptive (T cell) and innate (NK, NKT cell) anti-tumor immune responses. Indeed, much research has focused on the application of DC-based vaccines as a therapy for a variety of tumor types and several clinical trials are underway. DC-based vaccines for metastatic melanoma using cytokine activated, antigen-pulsed DC have in particular shown promising results (e.g., Cornforth, Lee & Dillman, 2011). Nevertheless, the complexity of the immune system makes it important to ensure that cellular therapies possess functional properties that lead to tumor cell lysis. For example, immature DCs have the potential to inhibit, rather than stimulate, tumor immunity in vivo. Accordingly, there is a great need for compositions comprising fully mature dendritic cells and other activated immune cells for treatment of conditions that may benefit from immunostimulation, such as cancer therapy.

### SUMMARY OF THE INVENTION

### Summary of Certain Embodiments

The invention relates to activated immunostimulatory cell compositions, methods of preparing those compositions, and to uses of the compositions to treat conditions that may benefit from immunostimulation, such as cancer.

Accordingly, in one aspect the invention is directed to methods for making an activated immunostimulatory cell composition, comprising: (a) incubating human leukocytes under conditions of time and temperature to activate the leukocytes; (b) subjecting the activated leukocytes to hypo osmotic shock; (c) adding to the leukocytes a salt solution in an amount effective to restore isotonicity; (d) mixing the leukocytes with a supportive medium; and (e) incubating the leukocytes in the supportive medium for a period of time to at least induce maturation of dendritic cells, thereby making an activated immunostimulatory composition.

In still another aspect, the invention is directed to methods of making an activated immunostimulatory cell composition comprising incubating non-quiescent (i.e., at least partially activated) leukocytes in a supportive medium under conditions of time and temperature that induce maturation of dendritic cells, thereby making an activated immunostimulatory composition.

In yet another aspect, the invention is directed to methods for making a composition comprising mature dendritic cells (DCs) comprising: (a) providing leukocytes, (b) allowing the leukocytes to transition from a quiescent to an active state by maintaining the leukocytes at room temperature for about 8 to 20 hours, (c) subjecting the leukocytes to hypo-osmotic shock, and (d) incubating the shocked leukocytes for 36 hours to 14 days in a supportive medium to thereby make a composition comprising mature DCs.

In some embodiments of these aspects of the invention, the leukocytes are incubated in the supportive medium for about 36 to 84 hours.

In some embodiments of these aspects of the invention, the leukocytes are incubated in the supportive medium for about 48-72 hours.

In some embodiments of these aspects of the invention, the composition is enriched in mature dendritic cells.

In some embodiments of these aspects of the invention, the composition further comprises activated lymphocytes.

In some embodiments of these aspects of the invention, the composition further comprises T helper cells enriched in Th1 phenotype.

In some embodiments of these aspects of the invention, the composition is enriched in Th1 cytokines.

In some embodiments of these aspects of the invention, the composition further comprises active macrophages enriched in the M1 phenotype.

In some embodiments of these aspects of the invention, the composition is enriched in M1 cytokines.

In some embodiments of these aspects of the invention, the leukocytes are isolated from peripheral blood, placental blood, cord blood, bone marrow, or lymphoid tissue.

In some embodiments of these aspects of the invention, the supportive medium is serum or plasma.

In some embodiments of these aspects of the invention, the supportive medium is serum or plasma that does not comprise exogenously added cytokines or interferons.

In some embodiments of these aspects of the invention, the methods further comprise adding at least one tumor antigen to the supportive medium.

In some embodiments of these aspects of the invention, the mature DC express at least one of HLA-DR, CD86, CD54, CD40, CD80, CD83 or CCR7 at levels higher than levels on a monocyte.

In some embodiments of these aspects of the invention, the methods further comprise removing the supportive medium and resuspending the leukocytes in a physiologically acceptable carrier.

In another aspect, the invention is directed to methods of making a cell-free composition, comprising a further step of collecting the supportive medium used in any of the various aspects for producing an activated immunostimulatory composition following incubation of the leukocytes, and removing the cells.

In another aspect, the invention is directed to compositions produced by any of the methods of preparing an activated immunostimulatory composition, including a cell-free portion of an immunostimulatory composition.

In still another aspect, the invention is directed to a composition comprising mature dendritic cells, activated helper T cells, cytolytic T cells, and at least one other leukocyte cell type.

In some embodiments of these aspects of the invention, at least 50% of the dendritic cells express at least one of HLA-DR, CD86, or CD54.

In some embodiments of these aspects of the invention, at least 5% of the dendritic cells express at least one of CCR7, CD40, CD80, or CD83.

In some embodiments of these aspects of the invention, at least 5% of the dendritic cells express CD8.

In some embodiments of these aspects of the invention, the composition comprises at least about 5 pg/mL IL-12.

In some embodiments of these aspects of the invention, the composition comprises at least about 1500 pg/mL IL-2.

In some embodiments of these aspects of the invention, the composition comprises at least about 100 pg/mL IFN-gamma.

In some embodiments of these aspects of the invention, the composition is depleted of cells.

In some embodiments of these aspects of the invention, the composition (including the cell-free composition) comprises at least about 5 pg/mL IL-12, at least about 1500 pg/mL IL-2, at least about 100 pg/mL IFN-gamma, or a combination of any two or three of these cytokines.

In one aspect, the invention is directed to methods of reducing the number of tumor cells in a subject having a tumor, comprising administering to the subject a composition of the invention, wherein the composition further comprises at least one antigen of the tumor.

In a similar aspect, the invention is directed to the use of any of the compositions of the invention in the preparation of a medicament for use in reducing the number of tumor cells in a subject having a tumor, wherein the composition further comprises at least one antigen of the tumor.

In a similar aspect, the invention is directed to compositions of the invention for reducing the number of tumor cells in a subject having a tumor, wherein the composition further comprises at least one antigen of the tumor.

In another aspect, the invention is directed to methods of stabilizing or regressing a tumor in a patient comprising: (a) collecting leukocytes from a patient afflicted with the tumor; (b) culturing the leukocytes at about 37° C. in a supportive medium that contains antigens from the patient's tumor but lacks exogenously added cytokines or growth factors to form mature dendritic cells and activated lymphocytes; and (c) administering a therapeutically effective amount of the composition to the patient.

In a similar aspect, the invention is directed to the use of any of the compositions of the invention in the preparation of a medicament for stabilizing or regressing a tumor in a patient.

In another similar aspect, the invention is directed to any of the compositions of the invention for stabilizing or regressing a tumor in a patient.

In some embodiments of these aspects of the invention, the administration is by systemic injection, intratumoral injection, or local injection into a lymph node draining the tumor.

In some embodiments of these aspects of the invention, the tumor is melanoma, metastatic melanoma, basal cell carcinoma, squamous cell carcinoma, Merkel cell carcinoma, breast cancer, colon cancer, rectal cancer, cervical cancer, oral cancer, Hodgkin's lymphoma, non-Hodgkin's lymphoma, sarcoma, cancer of the head and neck, esophageal cancer, bladder cancer, prostate cancer, or cancer of the peritoneal lining (mesothelioma).

In some embodiments of these aspects of the invention, the tumor is melanoma.

In still another aspect, the invention is directed to tumor vaccines comprising a composition of the invention, a tumor antigen, and an adjuvant.

Additional objects and advantages of the embodiments in the application appear in part in the following description and in part will be obvious from the description, or they may be learned in practice. The objects and advantages of the embodiments will manifest themselves by means of the elements and combinations particularly pointed out in the appended claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion only. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 shows dendritic cell (DC) markers on monocytes expressed as fold increase after incubation at 37° C. for 48 hours.

FIG. 2 compares expression of DC markers before and after incubation at 37° C. for 48 h in adherent and non-adherent culture conditions.

FIGS. 3A-3B compares expression of CD8 on monocytes before and after incubation at 37° C. for 48 hours. FIG. 3A presents % CD8 positive cells. FIG. 3B presents the Mean Fluorescence Intensity (MFI) of CD8.

FIGS. 4A-4B shows expression of the activation marker CD69 on lymphocyte subsets before and after incubation at 37° C. for 48 hours in the presence or absence of the superantigen, Staphylococcus aureus Enterotoxin B. FIG. 4A shows the % positive cells among all lymphocytes, T cells, or CD3 negative cells. FIG. 4B presents the Mean Fluorescence Intensity (MFI) of CD69 on positive cells in each population.

FIG. 5 shows expression of IL-2 receptor (CD25) on lymphocyte subsets before and after incubation at 37° C. for 48 hours in the absence (48 h) and presence (48 h SA) of superantigen, Staphylococcus aureus Enterotoxin B.

FIGS. 6A-6C shows expression of CD40 on monocytes before and after incubation at 37° C. for 48 hours in the absence and presence of superantigen, Staphylococcus aureus Enterotoxin B. FIG. 6A present the % CD40 positive cells. FIG. 6B presents the Mean Fluorescence Intensity. FIG. 6C shows the fold increase after 48 hour incubation.

FIG. 7A shows IL-12 production before and after incubation at 37° C. for 48 hours. FIG. 7B shows IL-12 production in the absence and presence of superantigen, Staphylococcus aureus Enterotoxin B, before and after incubation at room temperature (RT) or 37° C. for 48 hours.

FIG. 8 shows IL-2 content in AICC and IL-2 production by washed cells of AICC resuspended in fresh serum after incubation at 37° C. for 48 hours in the absence and presence of superantigen, Staphylococcus aureus Enterotoxin B.

FIG. 9 shows IFN-gamma production in the presence of superantigen, Staphylococcus aureus Enterotoxin B, after incubation at 37° C. for 48 hours and IFN-gamma production by washed cells resuspended in fresh serum after incubation at 37° C. for 48 hours in the presence of Enterotoxin B.

### DESCRIPTION OF THE EMBODIMENTS

As described in more detail below, the present invention relates to activated immunostimulatory cell compositions (AICCs), methods of preparing AICCs, and methods of using AICCs.

An AICC of the invention includes functionally active monocytes differentiated into mature DCs, as shown by their cell surface marker profiles, their ability to present antigens such as superantigens to T cells, and their release of IL-12, a key factor promoting preferential Th1 polarization. T cells in the AICC are also activated. The interaction of the mature DC with T cells in an AICC in the presence of antigen causes upregulation of IL-2 receptor on T cells and release of IL-2 and IFN-g. When DCs in an AICC are exposed to antigen, IL-12 production drastically increases. Accordingly, an AICC of the current invention is a powerful tool for immune stimulation. For example, when administered in vivo, an AICC can change the cytokine balance in the tumor environment to favor Th1 cytokines (e.g., interferons, IL-2), which activate proliferation of CTLs and cause tumor rejection.

Without being bound by theory, an AICC of the invention polarizes monocytes/macrophages into an M1 phenotype. As demonstrated in the working examples, the majority of monocytes/macrophages in AICC express high levels of HLA-DR and produce IL-12 and other M1 cytokines. M1 cytokines are known to overcome inhibitory effect of tumor environment on cellular immunity and promote tumor rejection. Accordingly, the cytokines in an AICC are useful in tumor therapies.

The principles and operation of the present invention may be better understood with reference to the drawings and accompanying descriptions. Before explaining at least one embodiment of the invention in detail, it is to be understood that the invention is not limited in its application to the details set forth in the following description or exemplified by the Examples. The invention is capable of other embodiments or of being practiced or carried out in various ways.

It is to be understood that the phraseology and terminology employed herein is for the purpose of description and should not be regarded as limiting. Further, the term “about” as used in connection with any and all values (including lower and upper ends of numerical ranges) includes a range of deviation of +/−0.5% to +/−20% (and values therebetween, e.g., ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%, ±10%, ±10.5%, ±11%, ±11.5%, ±12%, ±12.5%, ±13, ±13.5%, ±14%, ±14.5%, ±15%, ±15.5%, ±16%, ±16.5%, ±17%, ±17.5%, ±18%, ±18.5%, ±19%, ±19.5%, and ±20%).

### I. Methods of Making an Activated Immunostimulatory Cell Composition

Leukocytes require activation to mediate an immune response. As used here, an activated immunostimulatory cell composition refers to a composition comprising at least one type of activated leukocyte. In this context, “activated” means that a cell has acquired one or more functional or phenotypic characteristics of an activated cell. Examples of characteristics of an activated (or “matured”) dendritic cell include, but are not limited to, production of IL-12; absent or low level production of IL-10, expression of one or more of the costimulatory molecules CD80, CD86, CD83, CD40, or CD1c (BDCA1), of one or more adhesion molecules such as CD56, CD11b, CD11c, or IGSF4 (SynCam and Nectin-like-2), of one or more lectin receptors such as CLEC9A (DNGR-1), of one or more chemokine receptors such as CCR7, of one or more Toll receptors such as TLR1, TLR3, or TLR6, of one or more endocomal protein such as DC-LAMP, or of one or more transcription factors such as Id2, IRF8, or ICSBP; ability to activate naïve T cells via antigen presentation; and ability to induce B cell differentiation into antibody secreting (plasma) cells. Examples of characteristics of an activated T cell include, but are not limited to, production of one or more of IL-2, IFN-gamma, IFN-alpha, or IFN-beta; expression of IL-2R; upregulation of T cell activation markers such as one or more of CD69, CD71 (transferrin receptor 1), CD28, or CD40L; and proliferation following exposure to antigen, cytotoxic function, or helper function.

In one embodiment, an AICC is prepared from peripheral blood. Peripheral blood generally contains not only red blood cells (RBC) and platelets, but also leukocytes. Leukocytes, also known as “white blood cells,” include monocytes (a “precursor” cell that differentiates into macrophages of various tissues and dendritic cells), lymphocytes (which includes T cells, B cells, natural killer (NK) cells, and natural killer T cells (NKT cells)), and granulocytes (which includes neutrophils, basophils, and eosinophils).

Although whole peripheral blood is a convenient source of leukocytes, in alternate embodiments, an AICC is prepared using leukocytes isolated from blood from a central line, umbilical cord blood, placental blood, lymph, bone marrow, or lymphoid tissue such as lymph node or spleen. Leukocytes may be prepared by leukopheresis. Accordingly, the source of the leukocytes is not believed to be critical.

When whole blood is used, leukocytes can be partially separated from red blood cells and platelets by preparing a “buffy coat” using density gradient separation of the different cell types. Accordingly, in some embodiments, the amounts of platelets and red blood cells present in an AICC are lower than that in whole blood.

The starting materials for producing an AICC may be obtained from autologous or allogeneic sources. In one embodiment, an AICC is prepared from the patient who will ultimately be treated with the AICC; that is, the source is autologous. In other embodiments, an AICC is prepared from an individual other than the intended AICC recipient. In this case, the source is allogeneic.

In those embodiments, involving allogeneic starting materials, these may be conveniently obtained from a blood bank. The samples may be screened by the blood bank for blood type (ABO, Rh) or specific human leukocyte antigen alleles such as, but not limited to, A2, B12 and C3, irregular antibodies to red cell antigens, and transfusion-transmittable diseases. More specifically, screening can be conducted with antibodies using an Abbott PRISM instrument against: Hepatitis B, C, HIV 1/2, HTLV and Syphilis (−HCV; HbsAg; anti-HIV 1/2 O+; and anti-HTLV I/II). The samples can also be screened for HIV, HCV and HBV by molecular methods (NAT-Nucleic Acid Testing). Molecular screening can be accomplished using commercially available instrumentation, e.g., the TIGRIS system of Chiron or any other methods which may be suitable forms of testing for such diseases.

In one embodiment involving allogeneic sources, the sample is obtained from donors with the same blood type as the intended AICC recipient. In one embodiment, the donor(s) and recipient patient can be matched based on one or more HLA allele type. Alternatively, plasma samples can be obtained from donors with AB+ blood and the leukocytes can be obtained from donors with O-blood. Donors with AB+ blood are universal donors for plasma and donors with O-blood are universal donors for leukocytes. The plasma can be fresh, stored (e.g., at 1-6° C. for less than 24 hours), dried, or otherwise pre-treated (e.g., pathogen-reduced plasma and solvent/detergent (SD) treated plasma). Regardless of the source, all necessary processing of the sample(s) can be carried out without the need for highly specialized equipment.

In some embodiments, activated immunostimulatory cell composition may be prepared from smaller volumes of blood samples, with commensurate decreases in volumes of all solutions and use of smaller bags or other incubation vessels. Furthermore, use of these different size incubation vessels yields AICC with similar compositions. Use of smaller volumes provides the clinician with the ability to perform blood collection autonomously, without using an external blood bank. This may be useful when treating patients with otherwise healthy immune systems but suffering from some type of a small cancerous lesion.

In some embodiments, a method of preparing an AICC comprises a) activating human leukocytes; b) incubating the activated leukocytes in an incubation composition under conditions of time and temperature to induce differentiation and maturation of dendritic cells (DC), thus producing an AICC. In one embodiment, step (b) also induces activation of lymphocytes.

In one embodiment, the method further comprises contacting the DC with antigen or an antigenic peptide. In one embodiment, the antigen or antigenic peptide is contacted with the DC as they differentiate and mature in the incubation composition. That is, antigen or antigenic peptide is added during a part or all of the incubation of step (b). In one embodiment, the antigen or antigenic peptide is contacted with the DC after the incubation in the incubation composition is concluded. That is, the method further comprises a step (c) in which antigen or antigenic peptide is added to the AICC for a period of time sufficient to load DC with antigenic peptide.

In one embodiment, an Activated Leukocyte Composition produced using the methods of WO 2010/100570, is used in preparing the AICC. In this embodiment, the Activated Leukocyte Composition corresponds to step (a) of the above embodiment of the method.

In some embodiments, a method of preparing an AICC comprises a) isolating human leukocytes; b) optionally subjecting the leukocytes to hypo-osmotic shock; and c) incubating the shocked leukocytes in an incubation composition under conditions of time and temperature to induce differentiation and maturation of dendritic cells (DC), thus producing an AICC. In one embodiment, step (c) also induces activation of lymphocytes.

In one embodiment, the method further comprises contacting the DC with antigen or an antigenic peptide. In one embodiment, the antigen or antigenic peptide is contacted with the DC as they differentiate and mature in the incubation composition. That is, antigen or antigenic peptide is added during a part or all of the incubation of step (c). In one embodiment, the antigen or antigenic peptide is contacted with the DC after the incubation in the incubation composition is concluded. That is, the method further comprises a step (d) in which antigen or antigenic peptide is added to the AICC for a period of time sufficient to load DC with antigenic peptide.

In one embodiment, an Activated Leukocyte Composition produced using the methods of WO 2010/100570, is used in preparing the AICC. In this embodiment, the Activated Leukocyte Composition corresponds to steps (a) and (b) of the above embodiment of the method.

In some embodiments, the method comprises a) incubating human leukocytes under conditions of time and temperature to activate the leukocytes; b) optionally subjecting the leukocytes to hypo-osmotic shock; c) adding to the leukocytes of step b a physiologically acceptable salt solution in an amount effective to restore isotonicity; d) mixing the leukocytes of step c with a medium to form a second incubation composition; and e) incubating the second incubation composition under conditions of time and temperature to induce differentiation and maturation of dendritic cells (DC), thus producing an AICC. In one embodiment, step (e) also induces further activation of lymphocytes.

In one embodiment, the method further comprises contacting the dendritic cells (DC) of step (e) with antigen or an antigenic peptide. In one embodiment, the antigen or antigenic peptide is contacted with the DC as they differentiate and mature in the incubation composition. That is, antigen or antigenic peptide is added during part or all of the incubation of step (e). In one embodiment, the antigen or antigenic peptide is contacted with the DC after the incubation in the incubation composition is concluded. That is, the method further comprises a step (f) in which antigen or antigenic peptide is added to the AICC for a period of time sufficient to load DC with antigenic peptide.

In one embodiment, an Activated Leukocyte Composition produced using the methods of WO 2010/100570, is used in preparing the AICC. In this embodiment, the Activated Leukocyte Composition corresponds to steps (a) through (d) of the above embodiment of the method.

In some embodiments, the method comprises a) incubating human leukocytes at room temperature for up to about 20 hours to activate the leukocytes; b) subjecting the leukocytes to hypo-osmotic shock; c) adding to the leukocytes of step b a physiologically acceptable salt solution in an amount effective to restore isotonicity; d) mixing the leukocytes of step c with a medium to form a second incubation composition; and e) incubating the second incubation composition under conditions of time and temperature to induce differentiation and maturation of dendritic cells (DC), thus producing an AICC. In one embodiment, step (e) also induces further activation of lymphocytes.

In one embodiment, the method further comprises contacting the dendritic cells (DC) of step (e) with antigen or an antigenic peptide. In one embodiment, the antigen or antigenic peptide is contacted with the DC as they differentiate and mature in the incubation composition. That is, antigen or antigenic peptide is added during part or all of the incubation of step (e). In one embodiment, the antigen or antigenic peptide is contacted with the DC after the incubation in the incubation composition is concluded. That is, the method further comprises a step (f) in which antigen or antigenic peptide is added to the AICC for a period of time sufficient to load DC with antigenic peptide.

In one embodiment, an Activated Leukocyte Composition produced using the methods of WO 2010/100570, is used in preparing the AICC. In this embodiment, the Activated Leukocyte Composition corresponds to steps (a) through (d) of the above embodiment of the method.

In some embodiments, the method comprises a) activating human leukocytes; b) mixing the leukocytes of step a with a medium to form a second incubation composition; and c) incubating the second incubation composition under conditions of time and temperature to induce differentiation and maturation of dendritic cells (DC), thus producing an AICC. Activation of leukocytes is indicated by a change in expression levels or in the number of leukocytes expressing an activation marker of leukocytes, such as CD11b and/or CD62L. Accordingly, in one embodiment, activation of the leukocytes is indicated by increased expression of CD11b in the leukocyte population. Increased expression of CD11b can be detected, for example, by flow cytometry. Increased expression of CD11b encompasses an increase in the mean fluorescence intensity for CD11b on leukocytes, for example, the mean fluorescence intensity may be increased by at least about 5%, 10%, 15%, 20%, 25%, 30%, 35%, 40%, 45%, 50%, 55%, 60%, 65%, 70%, 75%, 80%, 85%, or 90%. Increased expression of CD11b also encompasses an increase in the percentage of leukocytes expressing CD11b (e.g., after correcting for background staining using an isotype control). For example, the percentage of leukocytes expressing CD11b may increase at least about 5%, 10%, 15%, 20%, 25%, 30%, 35%, 40%, 45%, 50%, 55%, 60%, 65%, 70%, 75%, 80%, 85%, or 90% relative to expression of CD11b expression on leukocytes in a buffy coat. In one embodiment, activation of the leukocytes is indicated by reduced expression of CD62L in the leukocyte population. Reduced expression of CD62L can be detected, for example, by flow cytometry. Reduced expression of CD62L encompasses a decrease in the mean fluorescence intensity for CD62L on leukocytes, for example, the mean fluorescence intensity may be reduced by at least about 5%, 10%, 15%, 20%, 25%, 30%, 35%, 40%, 45%, 50%, 55%, 60%, 65%, 70%, 75%, 80%, 85%, or 90%. Reduced expression of CD62L also encompasses a decrease in the percentage of leukocytes expressing CD62L (e.g., after correcting for background staining using an isotype control). For example, the percentage of leukocytes expressing CD62L may decrease at least about 5%, 10%, 15%, 20%, 25%, 30%, 35%, 40%, 45%, 50%, 55%, 60%, 65%, 70%, 75%, 80%, 85%, or 90% relative to expression of CD62L expression on leukocytes in a buffy coat. In one embodiment, CD62L and/or CD11b is measured on leukocytes that are granulocytes. In one embodiment, CD62L and/or CD11b is measured on leukocytes that are monocytes. In one embodiment, step (c) also induces further activation of lymphocytes.

In addition, in any of the other embodiments involving activation of leukocytes, changes in expression of CD11b and/or CD62L, as discussed above, may be used alone, together, or in combination with additional markers and assays, as discussed elsewhere in the application, as an indicator of leukocyte activation.

In one embodiment, the method further comprises contacting the dendritic cells (DC) of step (c) with antigen or an antigenic peptide. In one embodiment, the antigen or antigenic peptide is contacted with the DC as they differentiate and mature in the incubation composition. That is, antigen or antigenic peptide is added during part or all of the incubation of step (c). In one embodiment, the antigen or antigenic peptide is contacted with the DC after the incubation in the incubation composition is concluded. That is, the method further comprises a step (d) in which antigen or antigenic peptide is added to the AICC for a period of time sufficient to load DC with antigenic peptide.

In one embodiment, an Activated Leukocyte Composition produced using the methods of WO 2010/100570, is used in preparing the AICC. In this embodiment, the Activated Leukocyte Composition corresponds to steps (a) and (b) of the above embodiment of the method.

In general, in any of the methods of preparing an AICC, any composition in which the leukocytes have transitioned from a quiescent to a functionally active state can be used for the hypo-osmotic shock step. For example, as described in WO 2010/100570, leukocytes can be transitioned from a quiescent to functionally active state by incubating them at room temperature for up to about 20 hours. In one embodiment, the transition occurs by incubating the leukocytes for about 90 minutes to about 20 hours at room temperature. In one embodiment, the transition occurs by incubating the leukocytes for about 8 hours to about 20 hours at room temperature. In one embodiment, the transition occurs by incubating the leukocytes overnight at room temperature. In other embodiments, the temperature may be about 37° C. As noted, the details of this “transitioning” step are not essential and leukocytes may be obtained by any method for use in preparing an AICC.

Further, since hypo-osmotic shock is a type of stress, in those embodiments of the method that include a step of hypo-osmotic shock other methods may be employed to stress the cells. That is, since various types of stress elicit cellular responses through the same highly conserved signaling pathway consisting of protein kinase cascades that result in the activation of mitogen-activated proteins kinases (MAPKs), other stressors may be used in place of hypo-osmotic shock in any of the embodiments that mention hypo-osmotic shock. For example, in some embodiments, the methods of preparing an AICC comprise an optional step (in place of, or in addition to, hypo-osmotic shock) of subjecting the leukocytes to a stressor chosen from heat shock, hypoxia, treatment with any one or more of chlorpromazine, caffeine, vanadate, zymolyse, Congo red, calcofluor, rapamycin, or a mating pheromone, or by induction of actin depolymerization. Leukocyte activation also causes an increase in intracellular calcium, and there are many agonists that mimic this response. Accordingly, in still other embodiments, the methods of preparing an AICC comprise an optional step of subjecting the leukocytes to a calcium ionophore such as FMLP or PMA in place of or in addition to hypo-osmotic shock.

In any of the embodiments of the various methods of producing an AICC, an incubation under “conditions of time and temperature to induce differentiation and maturation of dendritic cells (DC)” (which may optionally also activate lymphocytes and other cells) generally is an incubation of from about 24 hours to about 14 days at from about room temperature to about 37° C.

In some embodiments, the incubation to induce differentiation and maturation of dendritic cells (DC) is at a temperature of about room temperature, i.e., in the range of about 12° C. to about 28° C. In one embodiment, the incubation is at a temperature of from about 16° C. to about 25° C. In one embodiment, the incubation is at a temperature of from about 18-25° C. In yet another embodiment, the incubation is at a temperature of from about 20-25° C.

In some embodiments, the incubation to induce differentiation and maturation of dendritic cells (DC) is at a temperature of about 37° C. In one embodiment, the incubation is at about 35° C. to about 38° C. In one embodiment, the incubation is at 37° C.+1-0.5° C.

In some embodiments, the time period of incubation to induce differentiation and maturation of dendritic cells (DC) is from about 24 hours to 14 days. Thus, in some embodiments the incubation is for about 24, 30, 36, 42, 48, 54, 60, 66, 72, 84, 96, 108, 120, 132, 144, 156, 168, 192, 216, 240, 264, 288, 312, or about 336 hours, or for about any range of hours in between these values.

In some embodiments, the time period of incubation to induce differentiation and maturation of dendritic cells (DC) is from about 48 to about 72 hours. In one embodiment, the incubation is for about 48 to about 72 hours at about 37° C. In one embodiment, the incubation is for about 48 hours at about 37° C. In one embodiment, the incubation is for about 72 hours at about 37° C. In one embodiment the incubation is for about 24 to about 72 hours at room temperature.

In some embodiments, the incubation is in a cell incubator in an atmosphere containing 5% CO2 and at 100% humidity. In some embodiments the incubation is in gas-permeable bags and the bags are placed in a cell incubator in an atmosphere containing 5% CO₂ and 100% humidity. In other embodiments, tissue culture flasks or dishes are used in the method. In still other embodiments, combinations of bags systems and culture dishes or flasks are used in the method. In those embodiments involving incubation in a bag system, the bag system may be one of those described in WO2010/100570, or it can be a bag made from a different material, such as but not limited to fluorinated ethylene propylene (FEP) or Ethyl Vinyl Acetate (EVA), or in a tissue culture vessel or in any vessel.

Any vessel used for incubation may also be treated or otherwise modified so that it becomes adhesive for leukocytes, which could be beneficial for leukocyte activation, differentiation of monocytes and activation/priming of lymphocytes. In one embodiment, one or more of the culture vessels used in the methods of preparing an AICC are non-adherent for dendritic cells. In another embodiment, the culture vessels used in the methods of preparing an AICC are treated to reduce cell adherence. In still another embodiment, one or more culture vessels used in the methods of preparing an AICC are treated to increase the adherence of cells.

Any vessel used for an incubation may also contain scaffolds. The scaffolds may be in different shapes and in particular could be microbeads, biodegradable or not biodegradable, e.g., made of collagen, or made of PLA, PGA (polylactic acid, polyglycolic acid) or similar synthetic polymers, hydrogel scaffolds made of gelatin, hyaluronic acid alginated, fibrin sealer. Scaffolds or bags could be coated with adhesion receptors, extracellular matrix proteins such as fibronectin or laminin or with active binding peptides from extracellular matrices, such as RGD. Scaffolds or microbeads could be also coated with activating stimuli or stimulating antibodies, such as but not limited to activating antibodies against CD3, CD28, or CD40. In at least one embodiment, however, the method does not comprise microbeads or scaffolds coated with one or more activating stimuli or with one or more antibodies against CD3, CD28, or CD40.

In some embodiments, the medium used for the incubation to produce an AICC is plasma or serum. In those embodiments utilizing serum, the serum may be obtained from a sample of plasma, which may be obtained from the same or a different whole blood sample (i.e., from the same or a different human) as the leukocytes, that has been contacted with a coagulating agent at about 37° C. In some embodiments, the serum or plasma is obtained from a commercial or non-profit supplier and may be either fresh or in a storage-compatible form, such as frozen.

In addition, although serum (particularly human serum) is often used in the incubation composition as the supportive medium, other supportive media may be used as well so long as it is a physiologic medium that supports release of cytokines, growth factors, and/or other soluble components from the activated leukocytes. For example, plasma may be used instead of serum. Other incubation medium that may also be used as supportive medium include culture medium, saline, or buffered saline solutions with optional addition of sugars and other components essential for cell viability and function such as amino acids (e.g. Lactated Ringer's solution, Acetated Ringer's solution, Hank's balanced salt solution (HBSS), Earle's balanced salt solution (EBSS), Standard saline citrate (SSC), HEPES-buffered saline (HBS), Gey's balanced salt solution (GBSS)). Saline solutions and culture medium may also be supplemented with human serum or clinical grade animal serum, or serum substitutes. The incubation composition may alternatively, or in addition, contain serum proteins such as human or bovine albumin, gamma-globulin, transferrin or other proteins from different tissues, plant proteins, or plant extracts.

In certain embodiments, leukocyte agonists such as complement proteins, chemokines, interferon-alpha, interferon-gamma, cytokines such as interleukin-4, granulocyte-macrophage-colony stimulating factor (GM-CSF), or interleukin-12, are added to the incubation. Monocyte differentiation to DCs in vitro can be induced using well-defined cytokine cocktails (Jensen S S, Gad M. 2010). Accordingly, in one embodiment, an incubation may be performed in the presence of cytokines such as interleukin-4 or GM-CSF. In other embodiments, an incubation may be performed with other substances that increase differentiation and maturation of dendritic cells and activation of lymphocytes and NK cells. For example, the CD40 co-stimulatory receptor on monocytes may be ligated by antibodies to CD40 or by a CD40 ligand (CD54) in the absence of cytokines (Brossart P, 1998). A CD40 independent activation of DC maturation can be induced by interaction with activated CD8 positive T cells (Ruedl C., 1999; Wirths, 2002). Accordingly, in one embodiment, exogenous, activated CD8 positive T cells may be added to the incubation. DC differentiation from monocytes in vitro can also be induced by DC interaction with NKT cells. DC differentiation results from NKT cell secretion of GM-CSF and IL-13, cytokines that were produced by the NKT cells upon activation by monocytes (Hegde, 2007). Accordingly, in one embodiment, exogenous, activated NKT cells may be added to the incubation. In other embodiments, DC differentiation and maturation may be promoted by the addition of one or more of GM-CSF, IL-4, IFN-gamma, IL-2, IFN-alpha, and TNF-alpha; and/or by addition of one or more bacterial products that interact with Toll receptors on DCs, such as but not limited to lipopolysaccharide (LPS), peptidoglycan (murein), double-stranded RNA or its synthetic analog polyinosinic:polycytidylic acid (poly I:C), Resiquimod (R-848), and Picibanil (OK-432).

It is also expressly contemplated that, in one or more embodiments of the methods, incubation occurs in the absence of one or more of the exogenously added factor(s) described above as involved in DC maturation. In one embodiment, all of the components needed for DC maturation are provided endogenously and no additional stimuli are added to the incubation composition. Nevertheless, various cytokines may be present in the incubation composition because they are released upon leukocyte activation during incubation. For example. CD40 ligand may be found in serum and on platelets that are part of the incubation composition. Similarly, activated CD8⁺ T cells and NKT cells that are endogenously present in the incubation composition can interact with monocytes to support dendritic cell differentiation and maturation.

Accordingly, in one embodiment, the incubation composition for producing an AICC does not include exogenous GM-CSF, exogenous IL-4, exogenous TNF, or an exogenous interferon (although one or more of GM-CSF, IL-4, TNF, or an interferon may be produced endogenously during the incubation). Thus, in one embodiment a method of preparing an AICC excludes the addition of one or more exogenous cytokine or interferon, the addition of reagent(s) that crosslink CD3 and/or CD28, the addition of reagent(s) that crosslink CD40, and/or the addition of other exogenous agents that promote dendritic cell maturation during the production of the AICC. Examples of exogenously added cytokines and exogenously added interferons that may be excluded from the practice of the method include any one or more of GM-CSF, IL-4, IFN-gamma, IL-2, IFN-alpha, or IL-2. Examples of exogenously added bacterial products that may be excluded from the practice of the methods include those known to interact with Toll receptors on DCs, such as but not limited to lipopolysaccharide (LPS), peptidoglycan (murein), double-stranded RNA or its synthetic analog polyinosinic:polycytidylic acid (poly I:C), Resiquimod (R-848), and Picibanil (OK-432).

In some embodiments inhibitors of angiogenesis targeting VEGF signaling are added to the incubation composition. These include but are not limited to anti-VEGF antibodies (e.g. bevacizumab, ranibizumab), antibodies against VEGF receptors (e.g. Brivanib, targets VEGFR-2 and FGFR), inhibitors of the tyrosine kinase activity of the VEGF receptors (e.g., Sorafenib, Cediranib, Sunitinib), soluble receptor-decoys (e.g, VEGF Trap, also called aflibercept), or vascular-disrupting agents (e.g., ZD6126).

In some embodiments adjuvants are added to the incubation composition. Examples of adjuvants include but are not limited to aluminium hydroxide, aluminium phosphate and calcium phosphate, adjuvants based on oil emulsions (Freund's emulsified oil adjuvants (complete and incomplete), Arlacel A, Mineral oil, emulsified peanut oil adjuvant (adjuvant 65), products from bacteria (their synthetic derivatives as well as liposomes) or gram-negative bacteria, endotoxins, cholesterol, fatty acids, aliphatic amines, paraffinic and vegetable oils, monophosphoryl lipid A, ISCOMs with Quil-A, and Syntex adjuvant formulations (SAFs).

As discussed elsewhere, in some embodiments, any of the methods may further comprise a contacting step wherein one or more antigen or antigenic peptide is introduced. Examples of antigens include tumor-specific and tumor-associated antigens, stem cell/cancer stem cell antigens, and superantigens (e.g., staphylococcal enterotoxins). Generally speaking, antigens or antigenic peptides will enhance differentiation of monocytes into dendritic cells and prime lymphocytes specific for that antigen. Further, since antigen presentation on the cellular level involves an antigenic peptide presented in the context of a class I or class II molecule, the terms “antigen,” “antigen peptide,” and “antigenic peptide” should not be construed as requiring contact with an intact antigen or a particular peptide. Instead, the terms are used broadly to indicate that an antigen presenting cell is contacted with antigenic material that it may then either directly, or after further processing, present in the context of class I or class II molecules.

Antigens and antigenic peptides, whether prepared from cell lysates or by recombinant expression of a protein or peptide, are incubated with an AICC or during the production of an AICC at various concentrations for about 1 hour to about 24 hours at room temperature to about 37° C. Examples of antigens/peptides include those listed below and any antigen/peptide used in the Examples section.

Recently, high-throughput technologies have enabled the identification of mutated gene in cancers. The number of these genes is high, with a functional heterogeneity broader than previously thought. (Stratton et al., Nature 458:719-24 (2009); Pleasance et al., Nature 463:191-96 (2010).) These studies have been performed in breast, colorectal, pancreatic, and lung cancers, as well as in glioblastomas, and overall have identified almost 400 candidate cancer genes (CAN-genes).

Some examples of shared antigens that are normally associated with spermatocytes or spermatogonia of the testis, placenta, and ovary include the cancer-testis (CT) antigens BAGE, GAGE, MAGE, NY-ESO-1, and SSX. These antigens are found in melanoma, lymphoma, lung, bladder, colon, and breast carcinomas. Shared antigens normally found in melanocytes, epithelial tissues, prostate, and colon also include the differentiation antigens Gp100, Melan-A/Mart-1, Tyrosinase, PSA, CEA, and Mammaglobin-A. These antigens are found in melanoma, prostate cancer, and in colon and breast carcinomas. Shared antigens that are ubiquitously expressed at low levels may be overespressed in cancers. Examples of overexpressed antigens include p53, HER-2/neu, livin, and survivin, found in esophagus, liver, pancreas, colon, breast, ovary, bladder, and prostate carcinomas. Other antigens are unique, such as β-catenin-m, β-Actin/4/m, Myosin/m, HSP70-2/m, and HLA-A2-R170J, which are associated with one or more of melanoma, non-small cell lung cancer, and renal cancer. Still other antigens are the tumor-associated carbohydrate antigens that are normally found in epithelia tissues such as renal intestinal and colorectal tissues. These antigens include GM2, GD2, GD3, MUC-1, sTn, abd globo-H, which can be found in melanoma, neuroblastoma, colorectal, lung, breast, ovarian, and prostate cancers.

Some additional exemplary antigen/peptides that may be used in the various aspects of the invention include MART-1, MAGE-1, MAGE-3, TYR, and gp100 antigens/peptides, which are associated with metastatic melanoma (e.g., as described in Butterfield et al., J. Immunotherapy 2008; 31:294-309; Markowicz et al., J Clin Oncol 27:15s, 2009 (suppl; abstr 9039)); TADG-12, CA125, hepsin, and other antigens/peptides associated with ovarian cancer (e.g., as described in U.S. Pat. Nos. 8,097,242 and 7,935,531); the carcinoembryonic antigen (CEA), which is associated with colorectal, gastric, and pancreatic carcinomas, some breast cancers, and many non-small cell lung cancers (e.g., as described in U.S. Pat. No. 8,012,468); antigens associated with neural cancers (e.g., glioblastoma multiforme and astrocytomas), such as the antigens tyrosine-related protein (TRP), melanoma-associated antigen-1 (MAGE-1), HER-2, AIM-2, IL-13 receptor alpha 2, or gp100 antigens and their peptide epitopes described in U.S. Pat. No. 8,097,256; hTERT (human telomerase reverse transcriptase), including the peptides described in U.S. Pat. No. 8,003,773; prostate specific antigen (PSA), prostate-specific membrane antigen (PSMA), and prostatic acid phosphatase (PAP) antigen, which are associated with prostate cancer (e.g., Tartour et al., Immunol Lett 2000; 74(1): 1-3); HPV (human papilloma virus) antigen (associated with cervical carcinoma); prostate specific G protein coupled receptor (PSGR) and six-transmembrane epithelial antigen of prostate STEAP described in U.S. Pat. No. 7,906,620 as associated with prostate and colon cancer; and/or PAGE4, which is associated with reproductive cancers such as prostate, uterine, and testicular cancer (as described in U.S. Pat. No. 7,910,692). In some embodiments, the intact antigen is used, whereas in other embodiments a peptide epitope of the antigen (prepared either by proteolytic digestion or recombinantly) is used.

In some embodiments, dendritic cells in an AICC are transfected with mRNA isolated from tumor or stem cells with known RNA sequence for tumor-specific antigens through electroporation, for example, using exponential decay wave or square-wave electroporators or other RNA pulsing apparatuses. In another embodiment, one or more antigen or antigen peptide is introduce into antigen presenting cells, such as dendritic cells in an AICC, using microparticle-based transfection, for example, as described in U.S. Pat. No. 8,097,243. In still other embodiments, one or more antigen or antigen peptide is introduced using adenovirus-based transduction, for example, as described in U.S. Pat. No. 8,012,468 and in Butterfield et al., J. Immunotherapy 2008; 31:294-309; or using a retroviral vector as described in U.S. Pat. No. 8,003,773.

In any of the embodiments, the method may result in one or more of differentiation of monocytes into maturate DC's, activation of lymphocytes, activation and/or proliferation of NK cells, or activation and/or proliferation of NKT cells.

In one embodiment, an AICC comprises “mature” DCs if the AICC includes cells that can stimulate activation (priming) of naïve T cells (as shown by expression of one or more of the T cell activation markers CD69, IL-2R, CD28, CD71, CD49d, CD40L, and/or by production of IL-2, IFN-alpha, IFN-beta, or IFN-gamma) and differentiation and proliferation of T helper and cytotoxic T cells in the presence of antigen. In another embodiment, the AICC comprises mature DC if there is an increase in production of IL-12. In another embodiment, the AICC comprises mature DC if there is an increase in the expression of one or more of the markers CD80, CD86, CD83, CD40, CD1c, CD56, CD11b, CD11c, IGSF4, CLEC9A, CCR7, TLR1, TLR3, TLR6, DC-LAMP, Id2, IRF8, or ICSBP on monocytes in the AICC. In one embodiment, an increase in expression is an increase in the number of one or more of molecules on the surface of a DC in the AICC (e.g., an increase in the mean fluorescence intensity (MFI) as determined by flow cytometry). An increase in the number of molecules is determined by comparing the MFI for that particular marker on a DC in the AICC that is suspected of being mature. In one embodiment, an AICC comprises “mature” DCs if there is an increase in the percentage of monocytes expressing one or more of the markers. In one embodiment, a monocyte is identified based on characteristic side scatter [SSC] and positive staining for the pan-leukocyte marker CD45 by flow cytometry, or by monocyte-specific CD14 staining. In one embodiment, the method results in an AICC in which both the MFI and the percentage of monocytes expressing at least one of the cell surface markers HLA-DR, CD54, CD86, CD83, CD80, CD40, and CCR7 increases. In one embodiment, the method results in an AICC in which both the MFI and the percentage of monocytes expressing any combination or all of the cell surface markers HLA-DR, CD54, CD86, CD83, CD80, CD40, and CCR7 increases. In one embodiment, an increase is assessed relative to the starting leukocyte composition used to produce the AICC (e.g., leukocytes in a buffy coat). In one embodiment, an increase is assessed relative to the cell composition used to being the incubation under conditions of time and temperature to induce differentiation and maturation of DC.

In one embodiment, an AICC will present antigens to naive T cells, causing the naïve T cells to differentiate into CD4 positive and CD8 positive cells, proliferate, produce IL-2, express IL-2 receptor, and produce interferons and other Th1 cytokines.

In another aspect, the methods may further comprise enriching an AICC of the invention for one or more cell populations. Compositions enriched for dendritic cells, T cells, NK cells, NKT cells, or other cell types can be prepared by cell sorting, panning, MACS, etc., using either positive or negative marker selection according to known methods.

In an additional embodiments, the methods may further comprise separating the cellular portion of the AICC from the liquid portion. In one embodiment, both the cellular and liquid (supernatant) portions are recovered. This may be accomplished, for example, by centrifuging the AICC and transferring the supernatant to a separate vessel. As described in the Examples, the supernatant is useful for therapy even in the absence of cells because of the cytokines and other soluble factors it contains. The cells in the pellet that forms following centrifugation may then be resuspended in any desired carrier. In other embodiments, the cells are removed from the liquid portion without recover of the cells, for example, by filtration. In still other embodiments, the cells are recovered without recovery of the supernatant, for example, by pelleting the cells and aspirating the supernatant.

In any of the embodiments, once an AICC is produced, the cells in the AICC may be isolated, either with or without additional concentration, and suspended in a carrier such as serum (which may be autologous or allogeneic with respect to recipient) or some other physiologically acceptable isotonically normal liquid suitable for storing and administering cells. Examples of such solutions are described below, and include solutions used to restore isotonicity, cell culture medium, buffered saline, or any other biocompatible fluid or specially formulated clinically acceptable cell storage or cell cryopreservation medium.

### II. Activated Immunostimulatory Cell Compositions

In another aspect, the invention relates to an Activated Immunostimulatory Cell Composition (AICC), which refers to any of the compositions produced by the methods of making an AICC described above. Accordingly, while “AICC” often refers to a cellular composition in the same carrier used in the incubation, as described above, an AICC also encompasses the cellular component in any carrier or excipient, as well as the liquid component separated from the cellular component.

Leukocytes in an AICC have certain characteristics that may be used to distinguish the individual leukocyte cell types or the composition as a whole. For example, monocytes in an AICC may express higher levels of CD54, HLA-DR and/or CD86 compared to freshly isolated monocytes. Further, monocytes in the AICC may express additional activation markers, such as one or more of CD8-alpha, CD83, CD80, CCR7, and/or CD40 compared to freshly isolated monocytes. An AICC composition may comprise a higher percentage or number of monocytes that have differentiated and matured into DCs than in a freshly prepared sample of, for example, peripheral blood leukocytes. Likewise, an AICC may contain a greater number or percentage of cells that are capable of activating/priming naïve lymphocytes than in a freshly prepared sample of, for example, peripheral blood leukocytes. Lymphocytes in the AICC may express higher surface levels of additional markers compared to freshly isolated lymphocytes, such as one or more of CD69, CD25, CD28, CD154, CD107a, and/or CD42d. In addition, leukocytes in the AICC may exhibit an increased ability to produce cytokines, such as one or more of IL-2, IFN gamma, IFN alpha, IFN beta, TNF alpha, TNF beta, and/or IL-12, compared to freshly isolated leukocytes.

As noted, in some embodiments of the method, an Activated Leukocyte Composition (ALC) produced using a method of WO 2010/100570 is used in the method of preparing the Activated Immunostimulatory Cell Composition (AICC). Although leukocytes in the ALC may be at least partially activated, as described in the Examples the leukocytes in the AICC may achieve higher levels of activation than in the ALC. The higher level of activation of leukocytes in an AICC compared to leukocytes in an ALC may be shown by any one or more of the characteristics noted above for an AICC using the ALC as the comparator.

As shown in the working examples, an AICC may also be characterized and distinguished from known compositions in terms of minimum activation level of DCs, e.g., as indicated by surface expression of HLA-DR, CD86, CD83, CD80, CD40, CD54, and CCR7 on monocytes; minimum activation level of lymphocytes, e.g., as indicated by surface expression of CD69, CD25 (IL-2R), CD28, CD154/CD40L, and CD49d; and minimum activation levels of NK and NKT cells, e.g., as indicated by surface expression of CD56, CD57, and CD107a. Surface expression of markers may be evaluated either as the percentage of cells expressing the marker or as the level of marker per cell.

In some embodiments, the final content of Activated Immunostimulatory Cell Composition (AICC) includes, in terms of the populations of leukocytes present, granulocytes, monocytes and lymphocytes. Specific amounts and relative percentages of the cells may differ based on the analysis techniques employed and on sample-to-sample variation. For example, when analysis is performed using a Cell Dyn Analyzer, the AICC generally contains about 45% to about 72% granulocytes (including neutrophils, eosinophils and basophils), about 3% to about 10% monocytes, and about 25% to about 50% lymphocytes. When analysis is performed using FACS (e.g., using a side-scatter versus a forward-scatter dot plot analysis or versus CD45 and/or CD14 fluorescence), the AICC generally contains about 50% to about 70% granulocytes; about 5% to about 15% monocytes, and about 15% to about 35% lymphocytes.

Granulocytes include neutrophils, eosinophils and basophils. In some embodiments, an AICC contains about 25% to about 85% neutrophils, about 0 to about 9% eosinophils; about 1.5 to about 4% basophils, about 2% to about 40% monocytes (including dendritic cells), and about 4% to about 70% lymphocytes, based on the total number of leukocytes in the AICC.

In any of the embodiments, an AICC may further contain residual levels of red blood cells, generally in the amount of about 0.05 to about 0.2 million per microliter, and/or residual levels of platelets, generally in the amount about 1 to about 100 thousand per microliter.

In some embodiments, the subpopulations of lymphocytes in the AICC are in the general ranges as follows: about 20% to about 80% T cells (CD3+); about 5% to about 40% B cells (CD19+); about 5% to about 35% NK cells (CD3−/CD56+), and/or about 0.1% to about 35% of NKT cells (CD3+/CD56+). In some embodiments, among T cells there are about 5% to about 65% T helper cells (CD4+/CD3+) and about 5% to about 75% cytotoxic T lymphocytes (CTLs, CD8+/CD3+).

In other embodiments, there about 40% to about 60% T cells (CD3+); about 15% to about 30% B cells (CD19+); about 15% to about 30% NK cells (CD3−/CD56+), about 2% to about 20% of NKT cells (CD3+/CD56+). In some embodiments, among T cells there are about 15% to about 40% of T helper cells (CD4+/CD3+) and about 25% to about 50% of CTL (CD8+/CD3+).

The ratio between Th cells and CTLs is usually about 0.5 to 1.5.

In any of the embodiments, the levels of DC, lymphocyte, NK, and NKT cell markers, as well as percentages of cells expression those markers, may be determined as described in the methods of preparing an AICC, or as described in the Examples.

In one embodiment, an AICC comprises DCs, wherein at least about 5%, 10%, 15%, 20%, 25%, or 30% of the DC express CD8, as detected by flow cytometry compared to an isotype control.

In one embodiment, at least about 1%, 5%, 10%, 15%, 20%, 25%, 30%, 35%, 40%, 45%, 50%, 55%, or 60% of the monocytes in the AICC are positive for the marker CCR7, as detected by flow cytometry compared to an isotype control.

In one embodiment, at least about 1%, 5%, 10%, 15%, 20%, 25%, 30%, 35%, 40%, 45%, 50%, 55%, or 60% of the monocytes in the AICC are positive for the marker CD40, as detected by flow cytometry compared to an isotype control.

In one embodiment, at least about 1%, 5%, 10%, 15%, 20%, 25%, 30%, 35%, 40%, 45%, 50%, 55%, or 60% of the monocytes in the AICC are positive for the marker CD80, as detected by flow cytometry compared to an isotype control.

In one embodiment, at least about 1%, 5%, 10%, 15%, 20%, 25%, 30%, 35%, 40%, 45%, 50%, 55%, or 60% of the monocytes in the AICC are positive for the marker CD83, as detected by flow cytometry compared to an isotype control.

In one embodiment, the mean fluorescence intensity (MFI) of total monocytes for the marker CD86 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CD86 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, the mean fluorescence intensity (MFI) of total monocytes for the marker CD83 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CD83 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, the mean fluorescence intensity (MFI) of total monocytes for the marker CD80 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CD80 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, the mean fluorescence intensity (MFI) of total monocytes for the marker CD40 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CD40 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, the mean fluorescence intensity (MFI) of total monocytes for the marker CCR7 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CCR7 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, the mean fluorescence intensity (MFI) of total monocytes for the marker CD54 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CD54 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, the mean fluorescence intensity (MFI) of total monocytes for the marker CD8 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher fresh than on peripheral blood monocytes. In one embodiment, the mean fluorescence intensity (MFI) of monocytes for the marker CD8 is at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 fold higher than on monocytes in an ALC prepared according to WO 2010/100570. In these embodiments, total monocytes are determined by SSC and staining for a pan-leukocyte marker.

In one embodiment, at least about 5%, 10%, 15%, 20%, 25%, 30%, or 35% of the CD3 positive lymphocytes in an AICC are positive for the marker CD69, as detected by flow cytometry compared to an isotype control, when the AICC is prepared in the presence of a superantigen. In one embodiment, the superantigen is Staphylococcal Enterotoxin B (SEB) at 100 ng/mL.

In one embodiment, the mean fluorescence intensity (MFI) of CD3 positive lymphocytes for the marker CD69 is at least about 0.5, 1.0, 1.5, 2.0, 2.5, 3.0, 3.5, 4.0, 4.5, or 5.0 fold higher when the AICC is prepared in the presence of a superantigen that mediates T cell-APC interaction. In one embodiment, the superantigen is Staphylococcal Enterotoxin B (SEB) at 100 ng/mL.

In one embodiment, at least about 5%, 10%, 15%, 20%, 25%, 30%, 35%, 40%, or 45% of the CD3 negative lymphocytes in an AICC are positive for the marker CD69, as detected by flow cytometry compared to an isotype control, when the AICC is prepared in the presence of a superantigen. In one embodiment, the superantigen is Staphylococcal Enterotoxin B (SEB) at 100 ng/mL.

In one embodiment, the mean fluorescence intensity (MFI) of CD3 negative lymphocytes for the marker CD69 is at least about 0.5, 1.0, 1.5, 2.0, 2.5, 3.0, 3.5, 4.0, 4.5, or 5.0 fold higher when the AICC is prepared in the presence of a superantigen that mediates T cell-APC interaction. In one embodiment, the superantigen is Staphylococcal Enterotoxin B (SEB) at 100 ng/mL.

In one embodiment, at least about 5%, 10%, 15%, 20%, 25%, 30%, or 35% of the CD3 positive lymphocytes in an AICC are positive for the marker CD25, as detected by flow cytometry compared to an isotype control, when the AICC is prepared in the presence of a superantigen. In one embodiment, the superantigen is Staphylococcal Enterotoxin B (SEB) at 100 ng/mL.

In one embodiment, the mean fluorescence intensity (MFI) of CD3 positive lymphocytes for the marker CD25 is at least about 0.5, 1.0, 1.5, 2.0, 2.5, 3.0, 3.5, 4.0, 4.5, 5.0, 5.5, or 6.0 fold higher when the AICC is prepared in the presence of a superantigen that mediates T cell-APC interaction than in the preincubation composition or in AICC incubated without superantigen. In one embodiment, the superantigen is Staphylococcal Enterotoxin B (SEB) at 100 ng/mL.

In one embodiment, at least about 5%, 10%, 15%, 20%, 25%, 30%, or 35% of the CD3 negative lymphocytes are positive for the marker CD25, as detected by flow cytometry compared to an isotype control, when the AICC is prepared in the presence of a superantigen. In one embodiment, the superantigen is Staphylococcal Enterotoxin B (SEB) at 100 ng/mL.

In one embodiment, the mean fluorescence intensity (MFI) of CD3 negative lymphocytes for the marker CD25 is at least about 0.5, 1.0, 1.5, 2.0, 2.5, 3.0, 3.5, 4.0, 4.5, or 5.0 fold higher when the AICC is prepared in the presence of a superantigen than in the preincubation composition or when it is prepared in the absence of a superantigen. In one embodiment, the superantigen is Staphylococcal Enterotoxin B (SEB) at 100 ng/mL.

In one embodiment, at least about 30%, 35%, 40%, 45%, 50%, 55%, 60%, 65%, 70%, 75%, or 80% of the monocytes in an AICC incubated with a superantigen are positive for the marker CD40, as detected by flow cytometry compared to an isotype control, when the AICC is prepared in the presence of a superantigen. In one embodiment, the superantigen is Staphylococcal Enterotoxin B (SEB) at 100 ng/mL.

In one embodiment, the mean fluorescence intensity (MFI) of monocytes for the marker CD40 is at least about 1.0, 2.0, 3.0, 4.0, 4.5, 5.0, 6.0, 7.0, 8.0, 9.0, or 10 fold higher than in the preincubation composition or when the AICC is prepared in the absence of a superantigen. In one embodiment, the T cell-APC superantigen is Staphylococcal Enterotoxin B (SEB) at 100 ng/mL.

In one embodiment, the concentration of IL-12 in an AICC is at least about 4, 5, 6, 7, 8, 9, 10, 11, 12, 13, 14, 15, 16, 17, 18, 19 or 20 picograms per milliliter, as determined by ELISA. In one embodiment, the concentration of IL-12 in an AICC incubated with a superantigen is at least about 0.5, 1.0, 1.5, 2.0, 2.5, 3.0, 3.5, 4.0, 4.5, or 5.0 fold higher when the AICC is prepared in the presence of a superantigen that mediates T cell-APC interaction than when it is prepared in the absence of a superantigen. In one embodiment, the superantigen is Staphylococcal Enterotoxin B (SEB) at 100 ng/mL.

In one embodiment, the concentration of IL-2 in an AICC is at least about 100, 500, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000 or more picograms per milliliter, as determined by ELISA. In one embodiment, the concentration of IL-2 is determined in an AICC incubated with a superantigen that mediates T cell-APC interaction. In one embodiment, the superantigen is Staphylococcal Enterotoxin B (SEB) at 100 ng/mL. In one embodiment, the superantigen is present during a 48 hour incubation at 37° C. used to produce an AICC.

In one embodiment, the concentration of IFN-gamma (IFN-g) in an AICC is at least about 80, 90, 100, 110, 120, 130, 140, 150, 160, 170, 180, 190, 200, 210, 220, 230, 240, 250, 260, 270, 280, 290, or 300 picograms per milliliter, as determined by ELISA. In one embodiment, the concentration of IFN-g in an AICC incubated with a superantigen is at least about 2.0, 2.5, 3.0, 3.5, 4.0, 4.5, 5.0, 5.5, 6.0, 6.5, or 7.0 fold higher when the AICC is prepared in the presence of a superantigen that mediates T cell-APC interaction than when it is prepared in the absence of a superantigen. In one embodiment, the superantigen is Staphylococcal Enterotoxin B (SEB) at 100 ng/mL.

In one embodiment, an AICC supports antigen presentation to naïve CD4 T cells, as shown by proliferation of the T cells when co-cultured with an AICC in the presence of antigen. In one embodiment, an AICC supports generation of T cells with a Th1 phenotype, as shown by the secretion of IL-2 and IFN-gamma by the T cells following co-culture with AICC.

In some embodiments, an AICC comprises T cell subsets in a ratio that is altered compared to the ratio of those same T cell subsets in peripheral blood. In one embodiment, an AICC comprises a ratio of CD4 T cells to CD8 T cells (CD4/CD8 ratio) that is less than about 1:1.

In one embodiment, an AICC has the characteristics of an AICC as described in the Examples with respect to one or more cell surface markers and/or cytokines. In one embodiment, an AICC has one or more of the characteristics of an “average” AICC presented in the Tables (that is, the characteristic reflects the mean+/−any standard deviation given in the Tables). In one embodiment, an AICC has one or more characteristics of a representative AICC as shown in the Figures, although any numbers in the Figures should be construed as “about” that number. In one embodiment, an AICC has a characteristic as described in the Examples following stimulation of the AICC with a superantigen.

In some embodiments, an AICC of the invention may be enriched for one or more cell populations. In one embodiment, an AICC is enriched for dendritic cells by negative selection of lymphocytes, for example, using anti-CD3 and anti-CD19 antibodies. In one embodiment, an AICC is enriched for lymphocytes by negative selection of monocytes and dendritic cells, for example using cell adherence or using anti-HLA-DR or anti-CD40, or anti-CD14 antibodies, or combinations thereof. In one embodiment, an AICC is enriched for T cells by positive selection, for example, using anti-CD3 antibody on coated beads in MACS or anti-CD2 antibody in FACS.

Any of the AICC may be used therapeutically in the inventive methods. But as described in detail elsewhere, in some embodiments an AICC is incubated with antigens from tumor cells produced from the patient's tumor tissue or tumor cell lines, or produced by recombinant methods or any other means of deriving or isolating antigens or antigenic peptides from a tumor, for example by eluting peptides from tumor cells. In some embodiments, the AICC, or the DCs within the AICC, is incubated with antigens common for cancer stem cells. In some embodiments, an AICC or DC within the AICC, is incubated with superantigens such as bacterial products. The addition of antigen/peptides will result in further maturation of DC from monocytes and more specific activation/priming of T cells present in the AICC.

An AICC may be separated into cellular and liquid components. Accordingly, in one embodiment, an AICC comprises the cellular and liquid components resulting directly from any of the methods. In one embodiment, an AICC comprises the cellular component separated from the liquid component, although the cellular component may be (re)-formulated in one or more carriers or excipients. In one embodiment, an AICC comprises the liquid component separated from the cellular component. It is believed that both the cellular components and the liquid components possess therapeutically beneficial properties. For example, the cellular component comprises matured DC and other cell types and the liquid component comprises various cytokines, such as IL-2 and IL-12.

In some embodiments adjuvants are added to an AICC to make a tumor vaccine. In one embodiment, the vaccine further comprises at least one tumor antigen/peptide. Examples of adjuvants include but are not limited to aluminium hydroxide, aluminium phosphate, calcium phosphate, Freund's complete adjuvant, Freund's incomplete adjuvant, Arlacel A, Mineral oil, emulsified peanut oil adjuvant (adjuvant 65), lipopolysaccharide (LPS), liposomes, endotoxins, cholesterol, fatty acids, aliphatic amines, paraffinic and vegetable oils, monophosphoryl lipid A, ISCOMs with Quil-A, and Syntex adjuvant formulations (SAFs).

In some embodiments, the vaccine may further comprise angiogenesis inhibitors. These include but are not limited to anti-VEGF antibodies (e.g. bevacizumab, ranibizumab), antibodies against VEGF receptors (e.g. Brivanib, targets VEGFR-2 and FGFR), inhibitors of the tyrosine kinase activity of the VEGF receptors (e.g., Sorafenib, Cediranib, Sunitinib), soluble receptor-decoys (e.g, VEGF Trap, also called aflibercept), or vascular-disrupting agents (e.g., ZD6126).

AICC compositions may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. It may also be accommodated by a notice associated with the container in a form prescribed by a governmental agency regulating the manufacture, use or sale of pharmaceuticals, reflecting approval by the agency of the form of the composition or human or veterinary administration. Such notice, for example, may be of labeling approved by the U.S. Food and Drug Administration for prescription drugs or of an approved product insert.

In an additional aspect, the invention provides an AICC formulated as a vaccine for treating a tumor. In one embodiment the vaccine further comprises at least one adjuvant as described above. In one embodiment, the vaccine comprises an AICC formulated with at least one of the tumor antigens described above. In one embodiment, the vaccine comprises an AICC that comprises matured dendritic cells, activated lymphocytes, at least 5 pg/mL IL-12, at least 1500 pg/mL IL-2, and at least 100 pg/mL IFN-gamma.

In another aspect, the invention provides an AICC for stimulating an immune response. The embodiments of this aspect include those described with respect to an AICC per se and those described with respect to the therapeutic uses of an AICC. For example, this aspect also relates to an AICC for treating a tumor or for stimulating an immune response to a tumor.

In an additional aspect, the invention provides for the use of any AICC in the preparation of a medicament for stimulating an immune response. The embodiments of this aspect include those described with respect to an AICC per se and those described with respect to the therapeutic uses of an AICC. For example, this aspect also relates to the use of an AICC for preparing a medicament for treating a tumor or for stimulating an immune response to a tumor.

### III. Therapeutic Uses

In another aspect, the invention provides methods in which an AICC is used as an immunostimulatory composition. According, the invention encompasses methods of stimulating an immune response to at least one tumor antigen, comprising administering an AICC of the invention to a subject. Subject includes both human and veterinary subjects.

In one aspect, the invention is directed to stimulating an immune response to a tumor. “Tumor” refers to all neoplastic cell growth and proliferation, whether malignant or benign, and all pre-cancerous and cancerous cells and tissues.

An AICC may be administered to treat any type of tumor. Examples of malignancies amenable to treatment include but are not limited to skin cancer such as melanoma, basal cell carcinoma, squamous cell carcinoma, Merkel cell carcinoma, breast cancers, colon cancers, rectal cancers, cervical cancers, oral cancers, liver cancers, pancreatic cancers, localized lymphomas, such as Hodgkin's lymphoma and various types of non-Hodgkin's lymphoma; sarcomas, cancers of the head and neck, esophageal cancers, bladder cancers, prostate cancers, gastric carcinoma, epipharyngial carcinoma, sigmoid carcinoma, rectal carcinoma, breast carcinoma, pelvic carcinoma, endometrial carcinoma, and peritoneal lining (mesothelioma). The tumors treated may be at various stages (stage I-IV) and grades (grades 1-4) according to TNM (American Joint Committee on Cancer (AJCC) staging system) and histological classification respectively. For example skin cancer can penetrate the epidermis, dermis, or the subcutaneous tissue without or with regional lymph node involvement. It could be well-differentiated, moderately differentiated, poorly differentiated, and undifferentiated (high grade).

An AICC may be used to treat primary tumors, tumor metastases, as well as ulcers/lesions that arise in various malignant pathologies, such as the benign skin lesions associated with Kaposi's Sarcoma. In one embodiment, an AICC inhibits residual malignant cells after tumorectomy.

In some embodiments, an AICC is used directly in a method of stimulating an immune response to a tumor without further manipulation. In other embodiments, prior to administration an AICC is incubated (pulsed) with antigens from tumor cells produced from the patient's tumor tissue or from related tumor cell lines, with antigens common for cancer stem cells, such as CD166, CD133, nestin, CD44, CD24 and ALDH1, or with peptide antigens known to be associated with the patient's tumor type. Other examples of tumor antigens and peptide antigens for particular tumor types are described in the method of preparing an AICC. In still other embodiments, an AICC is incubated with one or more “superantigen,” such as bacterial products, prior to administration. Contacting an AICC with an antigen or superantigen prior to administration will result in further and more specific activation/priming of T cells present in the AICC.

Additional examples and details regarding addition of antigen and antigen peptides are presented in the description of methods of preparing an AICC.

In some embodiments adjuvants are added to the AICC prior to administration. Examples of adjuvants include but are not limited to aluminium hydroxide, aluminium phosphate, calcium phosphate, Freund's complete adjuvant, Freund's incomplete adjuvant, Arlacel A, Mineral oil, emulsified peanut oil adjuvant (adjuvant 65), lipopolysaccharide (LPS), liposomes, endotoxins, cholesterol, fatty acids, aliphatic amines, paraffinic and vegetable oils, monophosphoryl lipid A, ISCOMs with Quil-A, and Syntex adjuvant formulations (SAFs).

In some embodiments, angiogenesis inhibitors are added to the AICC prior to administration. These include, but are not limited to, anti-VEGF antibodies (e.g. bevacizumab, ranibizumab), antibodies against VEGF receptors (e.g. Brivanib, targets VEGFR-2 and FGFR), inhibitors of the tyrosine kinase activity of the VEGF receptors (e.g., Sorafenib, Cediranib, Sunitinib), soluble receptor-decoys (e.g, VEGF Trap, also called aflibercept), or vascular-disrupting agents (e.g., ZD6126).

In general, application of the activated immunostimulatory cells composition is accomplished by one or more administrations of an AICC. In one embodiment, an AICC is administered systemically. Examples of systemic administration include intravenous, intramuscular, intraperitoneal, subcutaneous, and intradermal injection. In one embodiment, an AICC is administered locally, for example, intratumorally or intradermally or subcutaneously around the tumor or lymph nodes. In one embodiment, an AICC is administered intranodally; that is, an AICC is injected into one or more lymph nodes associated with (for example, draining) a tumor.

In some embodiment, an AICC is administered at a single site. In other embodiments, an AICC is administered at multiple sites.

In some embodiments, an AICC is administered by injection using a suitable syringe and needle (e.g., a 2 ml syringe fitted with an 18G or 25G needle). In those embodiments in which an AICC is injected directly into the tumor and/or the tissue surrounding the tumor and/or to regional draining lymph nodes, administration of the AICC may be through a catheter or endoscopic device under surveillance of ultrasound, X-ray and other similar technologies. In some embodiments, injection occurs about every one centimeter to about every three centimeters for the entire length of the tumor. In another embodiment, the injection is into healthy tissue surrounding the tumor, for example, tissue associated with lymph nodes draining the tumor. In one embodiment, injection occurs about every one centimeter to about every three centimeters in the area of healthy tissues associated with lymph nodes draining the tumor.

For injection, an AICC may be used directly. In this embodiment the incubation composition, which may contain cytokines that may help stimulate a tumor killing immune response, is administered along with the cellular portion of the AICC. In alternate embodiments, the liquid portion of the AICC may be removed, for example by centrifugation, and the cellular portion of an AICC then formulated in an aqueous solution (optionally more concentrated than the AICC), for example, in physiologically compatible buffers such as Hank's solution, Ringer's solution, or other physiological salt buffer, or in serum or plasma, including serum or plasma from the patient.

In another embodiment, an AICC is absorbed onto a physiologically inert and/or resorbable matrix or scaffold (e.g., collagen) and inserted by means of a press fit, into the lesion. This allows for a sustained delivery of the AICC into the site which benefits the patient in that the cells have a longer period in situ.

Depending on the severity and responsiveness of the condition to be treated, dosing can be of a single or a plurality of administrations, with course of treatment lasting from several days to several weeks, or until diminution of the disease state is achieved. Accordingly, in one embodiment an AICC is administered only once. In another embodiment, an AICC is administered at least two, three, four, five, or up to ten times or more. When the administration comprises at least two administrations, each administration may be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. Alternatively, each administration may be about one, two, three, four, five, or six months apart.

An AICC may be administered more than once if a clinician determines another application is necessary. Factors that may be taken into account include tumor size, spread of the tumor into surrounding tissues or draining lymph nodes, ulceration, suppuration, pyrexia or any other sign or symptom indicating an infection, or any clinical test(s) suggesting that re-treatment is warranted. In addition to re-treatment, referral for surgical treatment may be indicated at any point the clinician deems appropriate.

In the case of multiple administrations, the individual administrations may all be via the same route, or different routes of administration may be utilized for different administrations during the course of therapy.

The amount of an AICC to be administered will, of course, be dependent on the individual being treated, the severity of the affliction, the manner of administration, the judgment of the prescribing physician, etc. The dosage and timing of administration will be responsive to a careful and continuous monitoring of the individual's changing condition. Further, treatment algorithms should not be limited by the severity or type of malignancy, since an AICC may be more efficacious in patients presenting with advanced stage and poorly differentiated cancers.

In one embodiment, an AICC is used as a single treatment modality, where it may be administered once or more than once. In other embodiments, however, the AICC is administered as part of a combined treatment approach. In those embodiments involving combination therapy, an AICC is administered before, after, or in combination with other treatment modalities. An AICC may be used alone or in conjunction with any other conventional treatment for the specific type of cancer. Examples of conventional treatments include, but are not limited to radiation, chemotherapy, surgical tumorectomy, tumor oblation by electrophysiological methods such as laser treatment and photodynamic therapy, local, regional, and whole-body hyperthermia, therapy with angiogenesis inhibitors (such as anti-VEGF antibodies (e.g. bevacizumab, ranibizumab), antibodies against VEGF receptors (e.g. Brivanib, targets VEGFR-2 and FGFR), inhibitors of the tyrosine kinase activity of the VEGF receptors (e.g., Sorafenib, Cediranib, Sunitinib), soluble receptor-decoys (e.g, VEGF Trap, also called aflibercept), or vascular-disrupting agents (e.g., ZD6126)), therapy with cytokines such as IL-2 and interferon alpha, therapy with adjuvants such as BCG, therapy with antibodies such as Rituxan (for treatment of non-Hodgkin's lymphoma) or Trastuzumab (for treatment of breast cancer), therapy with G-CSF (Neupogen), erythropoietin (Epogen) and IL-11 to increase white, red and platelets cell counts respectively, bone marrow or hematopoietic stem cell transplantation, gene therapy, and therapy with molecularly targeted drugs.

Stimulation of an immune response against a tumor by an AICC can be demonstrated in various ways. For example, in one embodiment, administration of an AICC to a subject causes a reduction in the size of the tumor. Tumors may be evaluated for length, width, and height measurements and a 10%, 20%, 30%, 40%, 50%, 60% or more decrease in the sum of the products of the measurements indicates a reduction in tumor size. Alternatively or in addition, in one embodiment administration of an AICC to a subject inhibits progression of any preexisting lesions so that the tumor remains localized or encapsulated with no metastases. In still other embodiments, an AICC stimulates an immune response to a tumor when clinical tests usually used to monitor the status and/or progression of a particular tumor, such as X-rays, CT scans, MRIs, PET and PET/CTs, ultrasound, LDH testing, photoacoustic detection, or the level of a tumor biomarker (such as PSA for prostate cancer), demonstrate improvement or remission.

In any of the embodiments related to tumor therapy, responsiveness to the tumor therapy is measured by decreased serum concentrations of tumor specific markers. In certain embodiments, responsiveness to the tumor therapy is measured by one or more of an increased overall survival time, an increased progression-free survival, a decreased tumor size, a decrease in bone turnover metastasis markers, an increased impact on minimal residual disease, an increased induction of antibody response to the cancer cells that have been rendered proliferation-incompetent, an increased induction of delayed-type-hypersensitivity (DTH) response to injections of autologous tumor, or an increased induction of T cell response to autologous tumor or candidate tumor-associated antigens.

Irrespective of the nature of the treatment, an AICC may be useful in improving disease outcomes in patients. In addition, an AICC may also provide an analgesic effect.

### EXAMPLES

Reference is now made to the following examples, which together with the above descriptions illustrate the invention in a non-limiting fashion.

Unless otherwise noted, the nomenclature used and the laboratory procedures utilized include standard techniques. See, for example, “Current Protocols in Molecular Biology” Volumes I-III Ausubel, R. M., ed. (1994); “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); “Animal Cell Culture” Freshney, R. L, ed. (1986); “Methods in Enzymology” Vol. 1-317, Academic Press; and Marshak et al., “Strategies for Protein Purification and Characterization—A Laboratory Course Manual” CSHL Press (1996).

### Example 1

### Preparation of Exemplary Activated Immunostimulatory Cell Compositions (AICC)

Exemplary AICC were prepared as follows.

As an initial step, an activated leukocyte composition was prepared in accordance with the methods described in WO2010/100570. Briefly, buffy coat derived from a single unit of blood was incubated for 8-12 hours at room temperature. The cells were then subjected to hypo-osmotic shock (“HOS”) by addition of water for 40-45 sec, and isotonicity restored by adding a NaCl solution. The cells were pelleted by centrifugation and the cell pellet was resuspended in 50 mL serum obtained from the plasma fraction derived from the same blood unit. The leukocyte suspension in serum was then incubated for 90 min at 37° C. The serum was then discarded and fresh serum added to the leukocytes to make a final concentration of 3-4 million/mL. This initial composition is referred to in the examples as the “Preincubation Composition” (PC).

Although the examples utilize a PC that was incubated in serum for 90 min at 37° C., it is expressly contemplated that leukocytes pelleted after hypo-osmotic shock may also be resuspended in, for example, serum, and directly incubated for a period of time, e.g., 48 hours, to form the AICC.

In the current examples, the PC was concentrated by gentle centrifugation, removal of excess serum, and gentle resuspension of the leukocyte pellet in a volume of serum so that the leukocyte concentration was approximately 10 million/mL. The composition was then incubated for 48 or 72 hours (as indicated in the particular example) in a cell incubator at 37° C., 5% CO₂ and 100% humidity. The incubation was performed in gas-permeable FEP bags.

The current examples present results for AICC prepared using a concentrated PC so that the resulting AICC is also concentrated. A concentrated AICC may be better suited for clinical applications since the more concentrated the cells the smaller the injection volume needed to administer a sufficient amount of cells. But a comparison using non-concentrated PC showed that the leukocyte concentration did not affect the leukocyte composition in the AICC, either as assessed by percentage of cell types or by expression levels of specific markers. Accordingly, it is also possible to use a non-concentrated PC and the examples should in no way be read as limited to an AICC prepared using a concentrated PC.

In the examples that follow, leukocytes in the Preincubated Composition (PC) were compared to leukocytes in the AICC prepared from the concentrated PC, unless otherwise clearly specified in the example.

### Example 2

### Cell Population Analysis of an Activated Immunostimulatory Cell Composition (AICC)

The cell composition of the AICC was compared to that of the PC using two different cell counting methods: differential cell count on a Cell-Dyn Ruby Hematology Analyzer (Abbott Diagnostics) and flow cytometry analysis on a FACSCalibur™ (BD Biosciences). The Cell Dyn counts compare cell populations present in the PC (“before incubation”) and in an AICC after incubation for 48 hours at 37° C. In the table, WBC denotes white blood cells, or leukocytes. Both the total WBC count and the percentage of leukocyte types in the WBC were determined. The numbers of red blood cells (RBC) and platelets in the sample were also determined. The results of the Cell Dyn counts are summarized in Table 1.

TABLE 1
Cell Dyn Hematology Analysis

| | WBC | % WBC | | | | | RBC | platelets |
|---|---|---|---|---|---|---|---|---|
| Sample | ×10⁶/mL | granulocytes | lymphocytes | monocytes | eosinophils | basophils | ×10⁶/mL | ×10³/mL |
| before | 10.4 ± 1.7 | 62.2 ± 8.4 | 22.3 ± 5.4 | 11.4 ± 4.2 | 2.3 ± 1.2 | 1.8 ± 1.3 | 0.1 ± 0.1 | 118 ± 232 |
| incubation | | | | | | | | |
| 48 h | 5.1 ± 1.1 | 54.2 ± 9.7 | 36.3 ± 10.7 | 5.2 ± 1.9 | 1.7 ± 2.0 | 2.7 ± 0.2 | 0.1 ± 0.02 | 44 ± 32 |
| incubation | | | | | | | | |

The data presented are mean±SD of 5 experiments each performed with a blood sample from a different blood donation. Each Cell Dyn Hematology Analyzer measurement was performed in triplicate and the average count was calculated.

The percentages of leukocyte subtypes present in the PC (“before incubation”) and in an AICC after incubation for 48 hours at 37° C. were also determined by flow cytometry. Sampled cells were stained with pan-leukocyte antigen CD45 conjugated to peridinin chlorophyll protein (PerCP). CD45 staining permitted better resolution of leukocyte populations. Flow cytometry was performed using FACSCalibur™ (BD Biosciences) and population and marker analysis was done using FACSDiva™ software (BD Biosciences). Leukocyte populations were gated based on SSC and FL3 (CD45-PerCP) signals.

The percentage of granulocytes, lymphocytes, and monocytes in the leukocyte population was then determined. The results of the flow cytometry analysis of leukocyte populations are presented in Table 2 as mean+SD of 5 experiments.

TABLE 2
Flow Cytometry Analysis of Leukocytes

| | % WBC | | |
|---|---|---|---|
| sample | Granulocytes | Lymphocytes | Monocytes |
| before | 62.2 ± 12.9 | 21.5 ± 5.5 | 9.7 ± 4.3 |
| incubation | | | |
| 48 h | 59.0 ± 11.0 | 25.7 ± 6.8 | 10.6 ± 4.8 |
| incubation | | | |

The two counting methods produce slightly different results. Nevertheless, each method provides an acceptable measure of leukocyte subsets. The Cell-Dyn system is often used in clinical applications. But flow cytometry, as described below, can be advantageously used to determine the expression levels of individual molecules on the surface of an individual cell. In general, the AICC differed little from the PC in terms of percentages of the leukocyte subsets. The Cell-Dyn counts, however, indicate that the total number of leukocytes decreases during incubation to prepare the AICC.

The lymphocyte subset gated as described above based on SSC and CD45-PerCP fluorescence was further analyzed by flow cytometry. To separate T and B lymphocytes, samples were stained with an anti-CD3 antibody conjugated to fluorescein isothiocyanate (FITC) and with an anti-CD19 antibody conjugated to allophycocyanin (APC). Lymphocytes that were CD3⁺/CD19⁻ were counted as T cells, while cells that were CD3⁻/CD19⁺ were counted as B cells. Samples were also triple-stained with anti-CD4-APC, anti-CD8-phycoerythrin (PE), and anti-CD3-FITC antibodies. CD3⁺ lymphocytes were then separated into CD4⁺ T helper cells and CD8⁺ cytotoxic T cells (CTLs). NK and NKT cells were identified by double staining with anti-CD3-FITC and a mixture of anti-CD56 and anti-CD16 antibodies conjugated to PE ((CD56+CD16)-PE antibodies). NK cells were identified as CD3⁻/(CD56+CD16)⁺ cells and NKT cells were identified as CD3⁺/(CD56+CD16)⁺ cells. The results of 4-7 experiments performed with AICC produced from different blood donations are summarized in Table 3 and presented as mean±SD.

TABLE 3
Lymphocyte Subset Analysis by Flow Cytometry.

| | cell types | | | | | | |
|---|---|---|---|---|---|---|---|
| | | | | | | | % B |
| | | | | | % NKT cells | % NK cells | cells |
| | % T cells | % Th cells | % CTL | CD4/CD8 | CD3⁺/ | CD3⁻/ | CD3⁻/ |
| conditions | CD3⁺ | CD3⁺/CD4⁺ | CD3⁺/CD8⁺ | ratio | (CD56 + CD16)⁺ | (CD56 + CD16)⁺ | CD19⁺ |
| before | 38.8 ± 10.6 | 27.5 ± 13.0 | 33.8 ± 15.1 | 1.1 ± 0.8 | 9.0 ± 7.5 | 20.5 ± 5.8 | 29.2 ± 6.5 |
| incubation | | | | | | | |
| 48 h | 48.4 ± 9.1 | 28.7 ± 10.9 | 36.5 ± 11.9 | 0.9 ± 0.5 | 10.7 ± 7.0 | 20.7 ± 5.9 | 22.7 ± 6.8 |
| incubation | | | | | | | |

These results demonstrate a statistically significant increase from 38.8% to 48.4% (p=0.18) in the percentage of T-cells (CD3 positive) in the AICC compared to the percentage of T cells in the starting composition.

### Example 3

### Activation and Differentiation of Dendritic Cells (DCs) Analysis of Dendritic Cell Markers on Monocytes

Differentiation of monocytes into dendritic cells (DC) was assessed by flow cytometry analysis of expression of the DC-specific markers HLA-DR, CD54, CD86, CD83, CD80, CD40 and CCR7 on monocytes. Monocytes were analyzed first in Preincubated Composition (PC) and then in AICC produced using 48 or 72 hour incubations in gas-permeable FEP bags. Sampled cells were double-stained with antibodies against each of the DC-specific markers and with an antibody to the pan-leukocyte antigen CD45 conjugated to peridinin chlorophyll protein (PerCP). The latter was used for better resolution of leukocyte populations. Anti-HLA-DR and anti-CCR7 antibodies were conjugated to Allophycocyanin (APC). The rest of the DC-specific antibodies were conjugated to Phycoerythrin (PE).

Cells from each time point were washed with FACS staining solution (PBS, 2% Normal Mouse Serum; 0.02% Sodium Azide), aliquoted at 0.5×10⁶/tube and incubated with appropriate monoclonal antibodies for 30 min at 4° C. in the dark. The second antibody (anti-CD45-PerCP) was added for 15 min at 4° C. in the dark. After incubation, the cells were washed, resusupended in PBS, and analyzed on a FACSCalibur™ flow cytometer (BD Biosciences). Cells stained with irrelevant but isotype-matched antibodies under the same conditions were used as negative controls. The results were analyzed by FACSDiva™ software (BD Biosciences). Leukocyte populations were distinguished based on SSC and FL3 (CD45 positive, red fluorescence) signals.

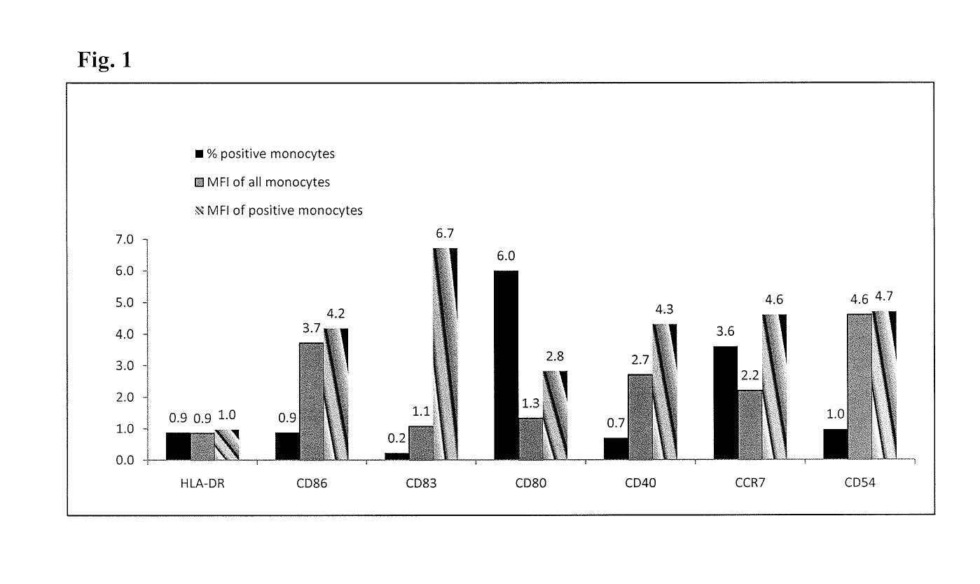

The results of flow cytometry analysis of DC markers on monocytes are summarized in Table 4 and a representative experiment is shown in FIG. 1.

TABLE 4
Analysis of DC-Specific Markers on Monocytes

| samples | HLA-DR | CD54 | CD86 | CD83 | CD80 | CD40 | CCR7 |
|---|---|---|---|---|---|---|---|
| before | 46618 ± 11003 | 6149 ± 656 | 3150 ± 721 | 5080 ± 203 | 442 ± 340 | 295 ± 189 | 208 ± 76 |
| after 48 h | 31930 ± 14702 | 33183 ± 10424 | 17689 ± 3492 | 1541 ± 1623 | 944 ± 432 | 1287 ± 1151 | 741 ± 315 |
| fold | 0.6 ± 0.3 | 5.6 ± 2.4 | 6.0 ± 1.8 | 2.9 ± 2.5 | 3.9 ± 3.9 | 4.7 ± 3.6 | 3.7 ± 1.8 |
| increase | | | | | | | |
| after 72 h | 47751 ± 23925 | 28262 ± 22334 | 14282 ± 290 | 1236 ± 492 | 905 ± 217 | 1910 ± 646 | 990 ± 57 |
| fold | 1.1 ± 0.4 | 5.0 ± 4.4 | 2.4 ± 3.1 | 0.8 ± 1.0 | 1.6 ± 1.8 | 5.4 ± 4.8 | 8.1 ± 6.5 |
| increase | | | | | | | |

There are two parameters that can be used to characterize marker expression: 1) percentage of cells expressing the marker (% positive cells), and 2) the Mean Fluorescence Intensity (MFI) of the marker, which depends on the number of marker molecules per cell. The data in Table 4 present the MFI of all monocytes for each marker, presented as mean+SD of 7-8 experiments. Fold increase was calculated for each marker in each experiment, then mean+SD calculated. As shown by the standard deviation, there is variability among experiments. Nevertheless, the MFI is several fold higher in the AICC (whether incubated for 48 hours or 72 hours) compared to the PC for most of the markers related to DC differentiation and maturation, confirming monocyte differentiation into DC during a 48 hour incubation of PC at 37° C.

FIG. 1 presents the fold increase for AICC prepared using a 48 hour incubation compared to the PC starting composition for a representative experiment. Fold increase is shown for three parameters for each marker: 1) the number of cells expressing a certain marker (% positive cells, first bar), 2) MFI for all monocytes (middle bar) and 3) MFI of “positive” monocytes (third bar). MFI represents the fluorescence distribution in the monocyte population. A measure of MFI for positive monocytes is included so that any increase in MFI in a population of cells that is a small percentage of total monocytes can still be detected. HLA-DR expression was already high in the preincubation composition (PC) used to prepare the AICC. In the PC, 94+3% of monocytes were HLA-DR positive and the MFI was also high. HLA-DR expression did not increase further in the AICC. For markers CD86, CD83, CD40, and CD54, the “% positive cells” remained about the same or even decreased as in the case of CD83 upon incubation (fold increase ˜0.2-1); however the MFI of all monocytes increased 2-5 fold mainly because the MFI of the positive cells increased many fold. For markers CD80 and CCR7 both % positive cells and MFI increased several fold.

These results show that AICC is enriched for mature DC compared to the starting composition.

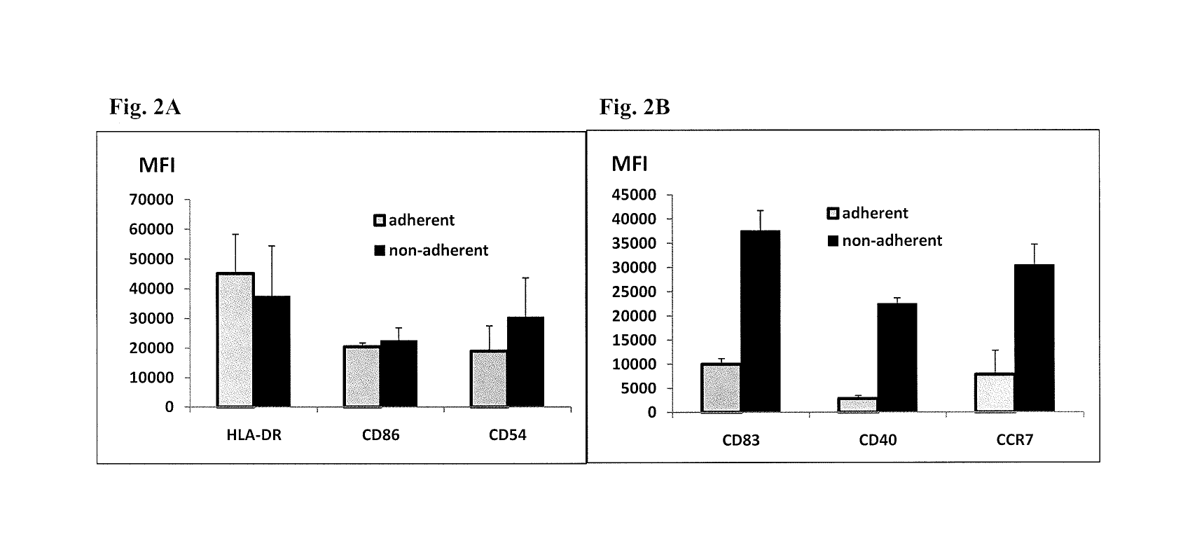

The effect of incubating cells under conditions that promote cell adherence was also investigated. AICC was prepared using two different types of bags, one type with a regular surface and the other type with a surface treated to promote cell adhesion. Expression of DC-specific markers was then compared for AICC prepared using the adherent and non-adherent bags. The results of this experiment are shown in FIG. 2. Surprisingly, incubation in non-adherent bags resulted in higher MFI for DC markers compared to incubation in adherent bags. The effect was particularly notable for CD83, CD40, and CCR7.

### Analysis of CD8-Alpha Expression on Monocytes

While monocytes express low levels of CD8-alpha, a subpopulation of differentiated DCs express high levels of this marker (Merad M, 2000). CD8α(+) dendritic cells (DCs) are important in vivo for cross-presentation of antigens derived from intracellular pathogens and tumors because they secrete high levels of IL-12 (Mashayekhi M, 2011) and promote a Th1 phenotype of T helper cells (Maldonado-López R, 1999; Maldonado-López R, 2001).

CD8 expression on monocytes was measured by flow cytometry. Monocytes were analyzed in the Preincubated Composition (PC) and in AICC produced using 48 hour incubations in gas-permeable FEP bags. Sampled cells were double-stained with antibodies against CD8 conjugated to PE and with an antibody to the pan-leukocyte antigen CD45 conjugated to PerCP. Leukocyte populations were distinguished based on SSC and FL3 (CD45) signals so that CD8 expression in the monocyte population could be analyzed.

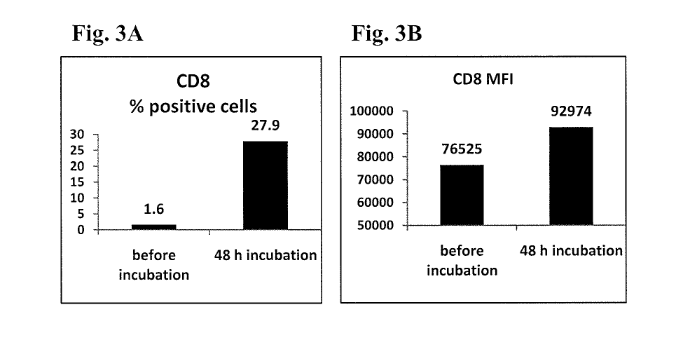

Table 5 summarizes the results of 3 experiments. FIG. 3 presents a representative experiment.

TABLE 5
Expression of CD8 on Monocytes

| conditions | % CD8⁺ monocytes | MFI |
|---|---|---|
| before incubation | 1.0 ± 0.6 | 65283 ± 9906 |
| 48 h incubation | 14.6 ± 15.2 | 71244 ± 23900 |

AICC prepared by incubation of the preincubation composition (PC) for 48 hours at 37° C. had an increased percentage of monocytes expressing CD8 compared to the percentage of CD8⁺ cells in the PC (Table 5 and FIG. 3A). The MFI of CD8 also increased (Table 5 and FIG. 3B).

### Example 4

### Effects of Superantigen Presentation by DC to T Cells in AICC

To confirm the functionality of DCs produced from monocytes during incubation of PC to produce AICC, Staphylococcal Enterotoxin B, a superantigen (SA), was added to the incubation mixture. Superantigens (SAs) resemble processed antigen peptides as they too engage MHC Class II molecules on antigen presenting cells and T cell receptor on T lymphocytes. SAs are advantageous, however, because unlike other antigens, they don't require intracellular processing. In addition, SAs stimulate about 20% of T cells bearing a certain family of T cell receptors; in contrast, most antigen peptides stimulate only around 0.001% of T cells because they only stimulate antigen-specific T cells. (Bhardwaj N, 1993). Thus, SAs can be used as a substitute for peptide antigen to test the ability of antigen presenting cells, such as DCs, to stimulate T cells.

### Analysis of T Cell Activation Markers CD69 and CD25 (IL-2R)

Activation of lymphocytes in AICC in the presence of SA was assessed by flow cytometry analysis of the expression of a well known lymphocyte activation marker, CD69. CD69 expression increases rapidly on activated T cells, with peak expression occurring 18-48 hours after stimulation. (Simms & Ellis 1996.) Lymphocytes were analyzed first in Preincubated Composition (PC) and then in AICC following 48 or 72 hours incubation in serum in cell incubator (at 37° C., 5% CO2 and 100% humidity) in the absence or presence of different doses of the SA Staphylococcal Enterotoxin B (SEB) (Sigma Aldrich). The PC was produced and concentrated as described above in the Example 1. Phytohemagglutinin (PHA), which is a polyclonal T cell stimulator that does not require interaction with antigen presenting cells such as DC, was used as a control.

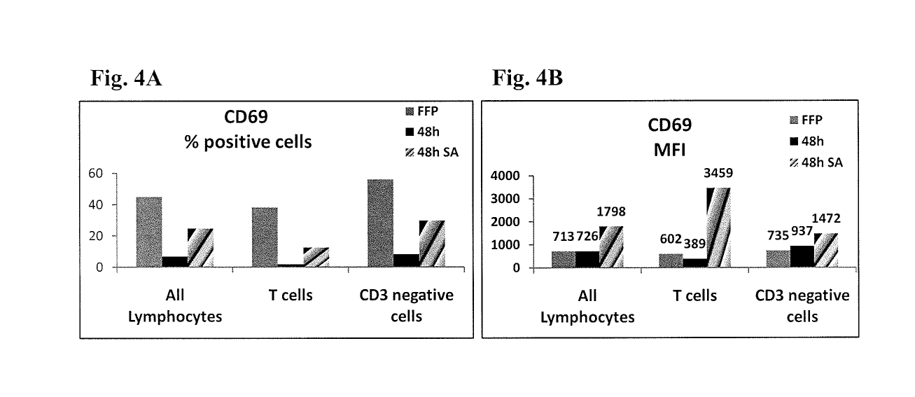

Lymphocytes were first double stained with anti-CD69-FITC antibody and anti-CD3-APC antibody (both from eBioscience), and then with anti-CD45-PerCP antibody. Cells were analyzed by flow cytometry. Mean+SD of 4 experiments is shown in Table 6. FIG. 4 presents a representative experiment.

TABLE 6
CD69 Expression on SA-Stimulated Lymphocytes

| | CD3pos CD69pos | | CD3 neg CD69pos | |
|---|---|---|---|---|
| | % pos | | % pos | |
| conditions | cells | MFI | cells | MFI |
| before | 11.4 ± 1.7 | 563 ± 132 | 32.9 ± 3.4 | 676 ± 91 |
| incubation | | | | |
| 48 h | 1.5 ± 1.2 | 550 ± 265 | 7.3 ± 5.2 | 711 ± 113 |
| incubation | | | | |
| 48 h SA10 | 8.9 ± 10.8 | 2157 ± 301 | 16.0 ± 14.0 | 1441 ± 932 |
| 48 h SA100 | 15.3 ± 13.2 | 2954 ± 30 | 26.7 ± 13.9 | 1883 ± 1056 |
| 48 h PHA | 7.1 ± 1.5 | 1389 ± 191 | 10.0 ± 1.6 | 649 ± 1 |

AICC at 48 hours contained a reduced number of CD69-positive T cells (CD3⁺) and non-T cells (CD3⁻) in the absence of SA. Addition of SA stimulated both subsets of lymphocytes, causing a dose-dependent increase in the percentage of CD69-positive cells (Table 6 and FIG. 4A) and in the Mean Fluorescence Intensity (MFI) of those cells (Table 6 and FIG. 4B). An increase in MFI reflects an increase in the number of CD69 molecules on each activated cell. The effect of SA was via a DC-dependent mechanism because phytohemagglutinin (PHA), an activator of lymphocytes that acts independent of antigen presentation by DCs, failed to produce similar effect, suggesting specific effect of SA via DC-dependent mechanism (Table 6).

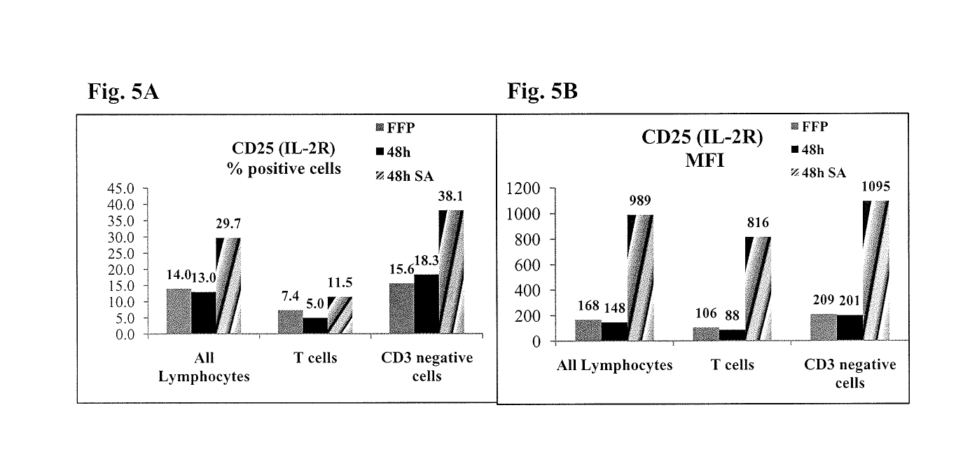

The effect of SA on expression of IL-2 receptor (CD25) was also studied. Antigen presentation causes release of IL-2 from lymphocytes and upregulation of IL-2 receptor on their surface resulting in amplification of the immune response. The preincubation composition was incubated in serum for 48 hours at 37° C. in the presence of 100 ng/mL SA. The resulting AICC was double stained with anti-CD25-PE antibody and anti-CD3-FITC antibody (both from eBioscience), and then with anti-CD45-PerCP antibody. The results of 4 experiments are summarized in Table 7 and a representative experiment is shown in FIG. 5.

TABLE 7
IL-2R Expression Following Superantigen Stimulation

| | CD25; all | | | | | |
|---|---|---|---|---|---|---|
| | lymphocytes | | CD25; T cells | | CD25; CD3 neg cells | |
| conditions | % positive | MFI | % positive | MFI | % positive | MFI |
| Before | 13.4 ± 3.8 | 123 ± 31 | 3.3 ± 2.3 | 80 ± 25 | 16.4 ± 6.5 | 158 ± 47 |
| incubation | | | | | | |
| 48 hour | 13.1 ± 3.9 | 162 ± 40 | 2.8 ± 1.4 | 81 ± 12 | 12.2 ± 7.3 | 236 ± 124 |
| incubation | | | | | | |
| 48 hour plus | 22.9 ± 11.5 | 550 ± 319 | 5.6 ± 4.0 | 529 ± 359 | 18.8 ± 14.3 | 606 ± 334 |
| SA 100 ng/ml | | | | | | |

Presentation of SA by DCs to lymphocytes increases the percentage of lymphocytes expressing IL-2R (Table 7, FIG. 5A) and the number of IL-2R on each cell (MFI) (Table 7, FIG. 5B). This effect was seen in both T cells and CD3-negative cells.

### Analysis of Monocyte Differentiation Marker CD40

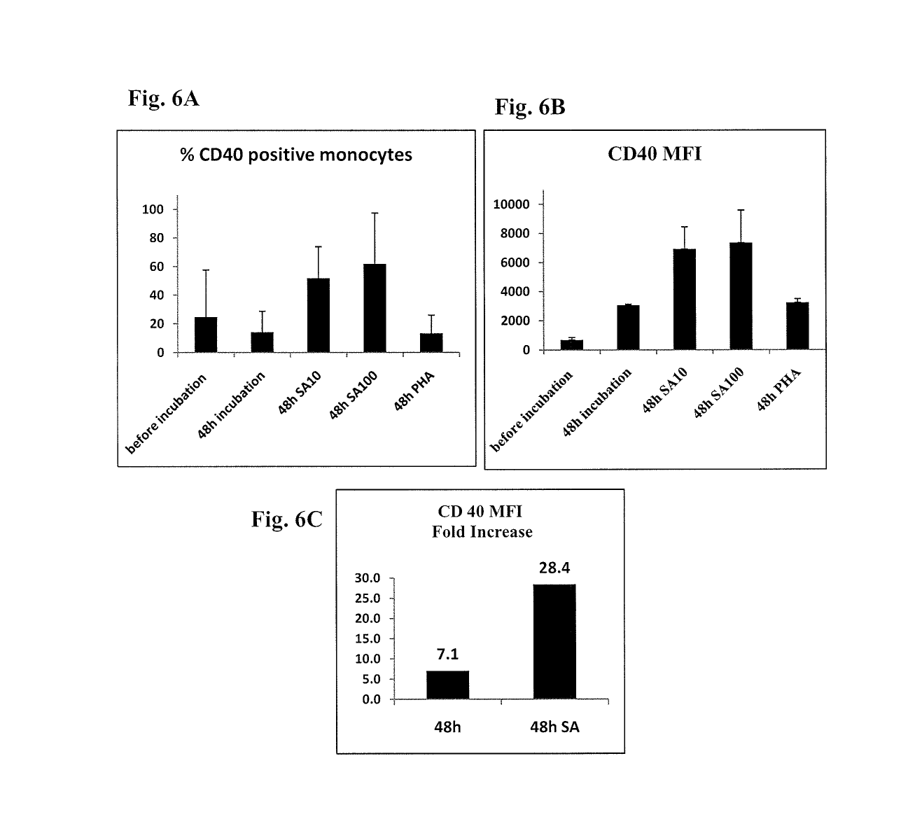

Addition of the superantigen (SA) during the 48 hour incubation used to make AICC not only promoted activation of lymphocytes but also stimulated further maturation of DCs. CD40 is a key co-stimulatory molecule on DCs that interacts with CD40L on T cells and induces production of IL-12 from DCs. Expression of CD40 therefore is an indication that DCs are functionally mature. To assess the maturation state of DC, levels of this marker were measured on monocytes in the preincubation composition and in AICC prepared either in the absence or presence of different amounts of superantigen. Table 8 and FIG. 6 summarize the results of 3 independent experiments performed with AICC from different blood donations.

TABLE 8
CD40 Expression on Monocytes

CD40 positive monocytes

| Conditions | % Positive | MFI |
|---|---|---|
| before incubation | 24.7 ± 33.0 | 683 ± 175 |
| 48 h incubation | 14.2 ± 14.6 | 3055 ± 74 |
| 48 h SA 10 ng/ml | 51.8 ± 22.2 | 6927 ± 1508 |
| 48 h SA 100 ng/ml | 61.9 ± 35.6 | 7358 ± 2239 |
| 48 H PHA 10 μg/ml | 13.3 ± 12.7 | 3237 ± 259 |

The percentage of CD40 positive cells in the AICC increased with increasing amounts of SA (Table 8 and FIG. 6A). Expression levels (MFI) of CD40 were also enhanced by addition of SA in dose-dependent manner (Table 8 and FIG. 6B). As shown by the MFI results, although levels of CD40 were high in AICC compared to the preincubation composition, the presence of SA resulted in even greater enhancement of CD40 levels on the cells (Table 8, “MFI” and FIG. 6C). Phytohemagglutinin (PHA), an activator of lymphocytes that acts independently of antigen presentation by DCs, failed to produce this effect.

### Analysis of IL-12 Content in AICC

When fully matured DCs interact with T cells, the DCs produce IL-12. Accordingly, the levels of IL-12 were also determined.

In the first experiment, the IL-12 concentration was measured in the PC and in AICC prepared using a 48 hour incubation at 37° C. in FEP bags. Ten mL of concentrated PC was added to a bag and cells were incubated for 48 hours to prepare AICC. PC and AICC compositions were centrifuged at 3,500×g for 30 min at 15° C. and IL-12 concentrations were measured in the supernatants with Diaclone™-ELISA for IL12 p70 (Gen-Probe, Inc) according to the manufacturer's instructions. Serum without addition of the cells, either obtained on the day of PC production or incubated simultaneously with AICC for 48 hours at 37° C., was centrifuged the same way. The OD values of the serum were used as a background and subtracted from the OD values of PC and AICC samples, respectively.

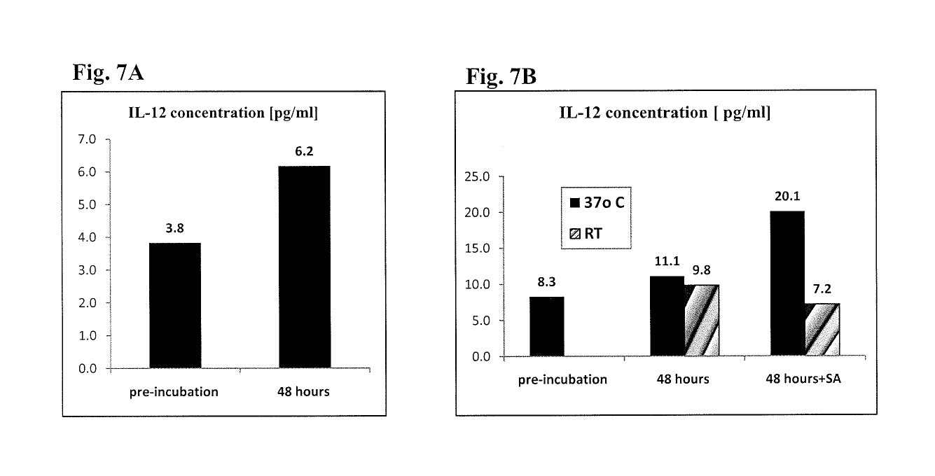

As shown in FIG. 7A, the IL-12 concentration was increased in the AICC. Accordingly, the results provide additional evidence that monocytes differentiated into DCs that are capable of producing IL-12 in the AICC. Considering that monocytes comprise only about 10% of all the leukocytes in AICC, these results are quite significant.

In the second experiment (FIG. 7B), PC was incubated at 37° C. for 48 hours in the absence and in the presence of 100 ng/ml SA to produce AICC. Parallel cultures were incubated at room temperature (RT). Again the IL-12 content increased during 48 hour incubation at 37° C. In the presence of SA, however, IL-12 concentration increased more than 2 fold. These results suggest that further maturation of DC occurs in the presence of SA. When incubation was performed at RT, no SA effect was seen, confirming that IL-12 release was a biological process dependent on cell function (FIG. 7B).

### Analysis of IL-2 Production by AICC Lymphocytes

Naïve T cells activated by DCs via antigen presentation acquire a CD4 positive Th1 phenotype and produce large amounts of the T cell mitogen cytokine IL-2. Accordingly, if the AICC included activated T cells, those T cells should release IL-2 in the presence of antigen since the AICC contains mature DC.

To detect IL-2 release, IL-2 concentrations were measured in the preincubation composition (PC) and in the AICC at the end of incubation for 48 hours at 37° C. in the presence and absence of 100 ng/ml SA. Samples were prepared as described for IL-12. After cell pelleting, IL-2 concentrations were measured in the supernatants using an IL-2 ELISA kit (eBioscience). As described above, pre-incubated and incubated serum samples were used as controls. In addition, the incubated cells (plus/minus SA) were pelleted, washed with culture medium, resuspended in fresh serum with no additives at 5×10⁶/mL, and placed in an incubator for 3 hours. The release of IL-2 into the fresh serum was then measured. The results of 3 experiments using different batches of PC as the starting composition are summarized in Table 9.

TABLE 9
IL-2 Release Following Superantigen Stimulation

| | IL-2 | IL-2 release into |
|---|---|---|
| | concentration | fresh serum |
| Conditions | [pg/mL] | [pg/mL/3 hours] |
| before incubation | below sensitivity | below sensitivity |
| 48 hour incubation | below sensitivity | below sensitivity |
| 48 hour incubation | 2562 ± 1206 | 131 ± 50 |
| with SA | | |

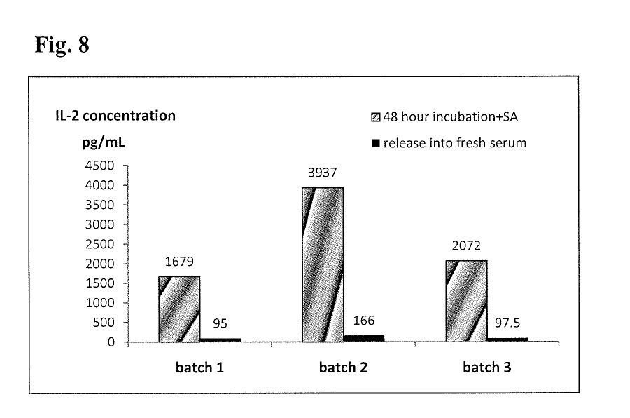

The data in Table 9 show that DC in AICC are functionally active and present SA to T cells causing robust release of IL-2. The results in Table 9 also demonstrate that the activated lymphocytes continue IL-2 release after SA and other biologically active agents in AICC have been washed off. As shown in FIG. 8, the concentrations of IL-2 in AICC produced in the presence of SA were high in all three samples derived from different subjects. There was a correlation between the concentration of IL-2 in incubated samples and the ability of lymphocytes to release IL-2 into fresh serum. Negligible amounts of IL-2 were produced when SA was not added to incubation medium (i.e., in the absence of antigen presentation).

### Analysis of IFN-Gamma Production by AICC Lymphocytes

CD4 T helper cells and CD8 cytotoxic T lymphocyte (CTL) effector T cells activated by DCs via antigen presentation produce large amounts of interferon gamma (IFN-g). IFN-g is a cytokine that is critical for innate and adaptive immunity and for tumor control. Accordingly, if the AICC included activated T cells, it should contain IFN-g.

IFN-g release during an incubation for 48 hours in the presence of SA was measured in the preincubation composition (PC) and in the AICC at the end of incubation for 48 hours at 37° C. in the presence and absence of 100 ng/ml SA. Samples were prepared as described for IL-12. The concentrations of IFN-g was measured in supernatants after cell pelleting using IFN-g ELISA kit (eBioscience). Pre-incubated and incubated serum samples were used as controls. The amount of IFN-g naturally present in the serum ranged from 1.5 to 5 pg/mL. These background serum concentrations were subtracted from the values obtained for experimental samples. In addition, the incubated cells (plus/minus SA) were pelleted, washed with culture medium and resuspended in fresh serum with no additives at 5×10⁶/mL. The cells were incubated for 3 hours and the release of IFN-g into the fresh serum was measured. The results of 4 experiments with different batches of PC are summarized in Table 10.

TABLE 10
IFN-g Release Following Superantigen Stimulation

| | IFN-g | IFN-g release into |
|---|---|---|
| | concentration | fresh serum |
| Conditions | [pg/mL] | [pg/mL/3 hours] |
| before incubation | below sensitivity | below sensitivity |
| 48 hour incubation | below sensitivity | below sensitivity |
| 48 hour incubation | 190.2 ± 86.6 | 39.5 ± 15.2 |
| with SA | | |

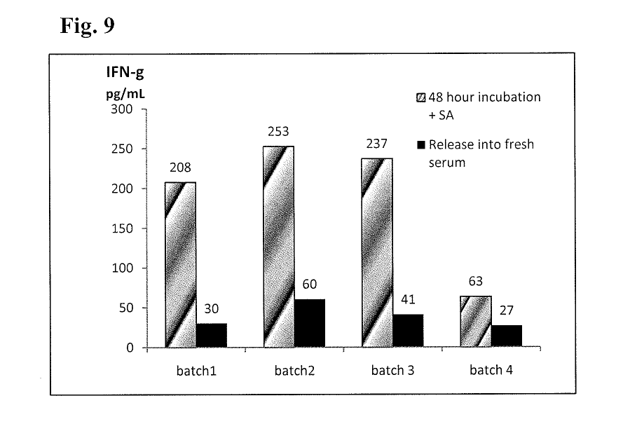

The data in Table 10 show that DC in AICC are functionally active and present SA to T cells causing robust release of IFN-g. The data in Table 10 also demonstrate that the activated lymphocytes continue to release IFN-g after SA and other biologically active agents in the AICC have been washed off. As shown in FIG. 9, there was a correlation between the concentrations of IFN-g in incubated samples and the ability of lymphocytes to release IFN-g into fresh serum. Negligible amounts of IFN-g are produced when SA is not added to incubation medium and no antigen presentation occurs.

### Example 5

### Expression of NK Cell Specific Activation Markers

Expression of two known NK cell activation markers, CD57 and CD107a, was also evaluated in the preincubation composition, and in AICC prepared by incubating for 48 hours at 37° C. in the absence and presence of SA. Leukocytes were stained with antibodies against CD57 conjugated to APC or with antibodies against CD107a conjugated to PE. A second antibody against CD14 marker on monocytes conjugated to FITC or APC respectively was added in order to achieve better resolution between leukocyte populations. Cells within a lymphocyte gate were analyzed. The results of two experiments are shown in Table 11.

TABLE 11
Expression of NK Markers on Lymphocytes
NK markers on Lymphocytes

| | CD57 lymph | | CD107a lymph | |
|---|---|---|---|---|
| conditions | % positive | MFI | % positive | MFI |
| before | 43.8 ± 1.3 | 14359 ± 5353 | 47.5 ± 2.6 | 420 ± 94 |
| incubation | | | | |
| 48 hour | 49 ± 1.0 | 16502 ± 190 | 46.6 ± 12.4 | 247 ± 75 |
| incubation | | | | |
| 48 hour | 42.6 ± 8.4 | 12798 ± 951 | 66.4 ± 12.1 | 363 ± 89 |
| incubation | | | | |
| with SA | | | | |

No significant differences were found in MFI for either marker. The percentage of CD107a positive cells increased (66.4% vs 47.5%) when incubation was performed in the presence of SA.

### Example 6

### Stimulation of an Immune Response Against Solid Tumors in Animal Models

An AICC can be evaluated in animal models to demonstrate that the soluble portions of the AICC stimulate an immune response to a tumor. Animal models for use in this assay include healthy mice with intact immune systems implanted with syngeneic tumor cells. One model uses the murine B16 melanoma cell line that is syngeneic to the C57BL/6 (H-2b) mouse strain. Another model that may be used is orthotopic or ectotopic implantation of 4T1 cells, which were originally derived from a spontaneous mammary tumor of a BALB/c mouse.

Briefly, an AICC is prepared as detailed above, for example, from blood donations of healthy human subjects. The AICC is produced by incubation for about 48 to 72 hours at 37° C. in the presence of a super antigen, such as 100 ng/mL Staphylococcus Entertoxin B. At the end of the incubation, cells in the AICC are removed, for example, by centrifugation, and the supernatant (i.e., the cell-free fraction of the AICC) is used for injection into mice bearing syngeneic tumors. After implantation, the cell-free AICC is administered to the animal one or more times. In one group of mice, the administration is systemic, while in another group the administration is by injection into the area around the tumor. An additional group of tumor bearing mice are administered AICC produced without superantigen. An additional group is administered human serum used for the incubation of activated leukocytes during production of the AICC. An untreated group serves as a control group.

Tumor size and animal survival are measured. Weight change and other cytotoxic effects are also monitored. The frequency of murine activated T cells producing IFN-gamma is measured by murine IFN-gamma enzyme-linked immunospot (ELISPOT) assay. A cell-free AICC is demonstrated to stimulate an immune response to a tumor if survival is prolonged, if tumor growth is inhibited, or if an animal administered an AICC exhibits an increased immune response compared to animals implanted with the tumor but not administered AICC or administered human serum alone.

### Example 7

### Treatment of Malignant Melanoma

The activated immunostimulatory cell composition is particularly useful for treatment of skin cancers and in particular melanoma. Melanoma is a malignant tumor of melanocytes. Melanocytes are cells that produce the dark pigment, melanin, which is responsible for the color of skin. They predominantly occur in skin, but are also found in other parts of the body, including the bowel and the eye. Although melanoma accounts for less than 5% of skin cancer cases, 75% of skin cancer deaths are due to melanoma. The incidence of melanoma has been steadily increasing for the past 30 years (American Cancer Society. 2009 Cancer Facts and Figures). In 2010, 114,900 new cases of melanoma were diagnosed in the US. (American Academy of Dermatology. Melanoma Fact Sheet. Accessed Nov. 1, 2010.)

To treat a melanoma, an AICC is prepared from a patient diagnosed with a melanoma. Alternatively an AICC is prepared from the peripheral blood of a healthy allogeneic donor, matched for blood type (ABO, Rh) and specific human leukocyte antigen (HLA) alleles such as HLA-A2, HLA-B12 and HLA-Cw7. These are the most frequent alleles among melanoma patients (Fensterle et al., BMC Med. 2006, 13; 4:5).

The AICC is stimulated (either during preparation or in a separate incubation afterward) with tumor antigens from the patient's melanoma biopsy or from tissue obtained from surgical resection of the tumor. Tumor cell lysates are prepared. Alternatively, primary melanoma cell lines derived from biopsy material are subjected to melanoma chemotherapeutic agents such as dacarbazine and temozolomide according to the protocol described in Naumann et al., Br J Cancer. 2009 Jan. 27; 100(2):322-33. Apoptosis of the treated cells is confirmed, for example by flow cytometry analysis for caspase 3 activity using the assay described in He et al., J Immunol Methods. 2005 September; 304(1-2):43-59). The melanoma lysates or apoptotic cells are then loaded onto DCs in the AICC.

AICC may also be stimulated with known melanoma antigens. Exemplary melanoma antigens include MART-1, MAGE-1, MAGE-3, TYR, and gp100 antigens.

The functional activity of cytotoxic T cells in AICC loaded with melanoma antigens is assessed in an ex-vivo cytotoxicity assay. For this assay, primary melanoma cell lines are established from the same biopsy or surgical material. Cells are isolated by enzymatic dissociation with collagenase and DNase and cell suspensions are cultured in Dulbecco modified Eagle medium 10% fetal calf serum (Gibco-BRL), HEPES (1:500), penicillin-streptomycin (1:100), and glutamine (1:100). Adherent cultured melanoma cells are incubated with AICC at various ratios for 6 hours at 37° C. Melanoma cells are then washed and analyzed by flow cytometry using caspase 3 activity for evidence of apoptosis.

The AICC composition is then aspirated into a sterile syringe of any size, using an 18-gauge (18G) needle. Aspiration is performed slowly to minimize damage to the cells. While the size of the syringe and needle are by no means limiting, a large gauge needle is preferred for aspiration. This facilitates the transfer and reduces cell damage.

The AICC is administered by injecting the composition into and/or intradermally around the tumor. The AICC may also be injected into regional lymph nodes for a systemic effect. Alternatively, the entire syringe can be injected at one time into a single site within the tumor. The clinician may choose to administer additional AICC if it is determined to be necessary based on clinical parameters.

The patient is monitored and the effect of the AICC on parameters such as tumor size, number of metastases, progression-free survival, immune response to tumor antigens, etc. are determined. The frequency of melanoma-specific CD8+ T cells in the patient's peripheral blood is monitored, for example, with IFN-gamma enzyme-linked immunospot (ELISPOT) assay.

### Example 8

### Preparation of Peptide-Pulsed AICC for Stimulating an Anti-Tumor Immune Response in a Cancer Patient

A tumor biopsy is obtained from a patient with a tumor, along with a sample of peripheral blood. Alternatively, peripheral blood may be obtained from a donor other than the patient, particular if the donor is matched for one or more HLA antigens with the patient. Peripheral blood may be obtained by standard phlebotomy/blood banking techniques, including leukopheresis. In those embodiments involving allogeneic starting materials, these may be conveniently obtained from a blood bank. The samples may be screened by the blood bank for blood type (ABO, Rh) or specific human leukocyte antigen alleles such as, but not limited to, A2, B12 and C3.

Tumor tissue is disrupted under sterile processing conditions to obtain tumor cells. Tumor cells are either then used immediately to prepare tumor antigen, cultured (passaged), or cryopreserved for later use. If desired, expression of one or more tumor antigen associated with the particular tumor type can be verified by flow cytometry, PCR, or other standard techniques.

An AICC is prepared from the patient's peripheral blood sample as described above. Antigen is added either during a part of the incubation to prepare an AICC, or following preparation of an AICC. The AICC is pulsed for at least an hour with tumor cell antigens prepared from the patient's tumor cells. If the tumor cell antigens are added during preparation of the AICC, then pulsing lasts for the duration of the AICC incubation. As an alternative to tumor cell antigens, one or more peptide antigen associated with the particular tumor type may be used, for example, at a concentration of about 10 microgram/ml (per peptide antigen). When a tumor antigen preparation or one or more peptide antigens is added following production of the AICC, the AICC is generally pulsed with the antigen or peptide antigen should for about 16-20 hours at 37° C. Because the AICC contains not only dendritic cells but also other cell types such as activated T lymphocytes as well as cytokines and other growth factors, the AICC may be further incubated at 37° C. to allow the DC to present the tumor antigen(s) or tumor peptide antigen(s) to T cells in the AICC and the cytokines to act to further promote activation of the cells.

Following incubation, the AICC is administered to the patient. The antigen-pulsed AICC may be infused, or it may be administered by injection at a site local to the tumor. When the antigen-pulsed AICC is injected, it may optionally be concentrated to reduce the volume so that the AICC contains about 10⁷ to 10⁸ DC. If the AICC volume is reduced for injection, it may be desirable to administer the AICC supernatant by a separate injection or by infusion.

Following administration of the AICC, the patient is monitored for tumor response.

All publications cited in the specification, including patent publications and non-patent publications, are indicative of the level of skill of those skilled in the art to which this invention pertains. All these publications are herein incorporated by reference to the same extent as if each individual publication were specifically and individually indicated as being incorporated by reference.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for making an activated immunostimulatory cell composition, comprising:
a) incubating human leukocytes under conditions of time and temperature to activate the leukocytes;
b) subjecting the activated leukocytes to hypo osmotic shock;
c) adding to the leukocytes a salt solution in an amount effective to restore isotonicity;
d) mixing the leukocytes with a supportive medium; and
e) incubating the leukocytes in the supportive medium for a period of time to at least induce maturation of dendritic cells, thereby making an activated immunostimulatory composition.

2. A method of making an activated immunostimulatory cell composition comprising incubating non-quiescent leukocytes in a supportive medium under conditions of time and temperature that induce maturation of dendritic cells, thereby making an activated immunostimulatory composition.

3. A method for making a composition comprising mature dendritic cells comprising:
a) providing leukocytes,
b) allowing the leukocytes to transition from a quiescent to an active state by maintaining the leukocytes at room temperature for about 8 to 20 hours,
c) subjecting the leukocytes to hypo-osmotic shock, and
d) incubating the shocked leukocytes for 36 hours to 14 days in a supportive medium to thereby make a composition comprising mature dendritic cells.

4. The method of claim 1, wherein:
(i) the leukocytes are incubated in the supportive medium for about 36 to 84 hours; or
(ii) the leukocytes are incubated in the supportive medium for about 48-72 hours.

5. (canceled)

6. The method of claim 1, wherein:
(i) the composition is enriched in mature dendritic cells;
(ii) the composition further comprises activated lymphocytes;
(iii) the composition further comprises T helper cells enriched in Th1 phenotype;
(iv) the composition is enriched in Th1 cytokines;
(v) the composition further comprises active macrophages enriched in the M1 phenotype; and/or
(vi) the composition is enriched in M1 cytokines.

7-11. (canceled)

12. The method of claim 1, wherein:
(i) the leukocytes are isolated from peripheral blood, placental blood, cord blood, bone marrow, or lymphoid tissue;
(ii) the supportive medium is serum or plasma;
(iii) the supportive medium is serum or plasma that does not comprise exogenously added cytokines or interferons;
(iv) at least one tumor antigen is added to the supportive medium;
(v) the mature dendritic cells express at least one of HLA-DR, CD86, CD54, CD40, COSO, CD83 or CCR7 at levels higher than levels on a monocyte; and/or
(vi) the method further comprises removing the supportive medium and resuspending the leukocytes in a physiologically acceptable carrier.

13-17. (canceled)

18. A method of making a cell-free composition, comprising collecting the supportive medium of claim 1 following incubation of the leukocytes, and removing the cells.

19. A composition produced by the method of claim 1.

20. A composition comprising mature dendritic cells, activated helper T cells, cytolytic T cells, and at least one other leukocyte cell type.

21. The composition of claim 20, wherein:
(i) at least 50% of the dendritic cells express at least one of HLA-DR, CDS6, or CD54;
(ii) at least 5% of the dendritic cells express at least one of CCR7, CD40, COSO, or CDS3;
(iii) at least 5% of the dendritic cells express CDS;
(iv) the composition comprises at least about 5 pg/ml IL-12;
(v) the composition comprises at least about 1500 pg/ml IL-2; and/or
(vi) the composition comprises at least about 100 pg/ml IFN-gamma.

22-26. (canceled)

27. The composition produced by the method of claim 18, wherein the composition comprises:
(i) at least about 1500 pg/ml IL-2; and/or
(ii) the composition comprises at least about 100 pg/ml IFN-gamma.

28. A method of reducing the number of tumor cells in a subject having a tumor, comprising administering to the subject a composition of claim 20, wherein the composition further comprises at least one antigen of the tumor.

29. A method of stabilizing or regressing a tumor in a patient comprising:
a) collecting leukocytes from a patient afflicted with the tumor;
b) culturing the leukocytes at about 37° C. in a supportive medium that contains antigens from the patient's tumor but lacks exogenously added cytokines or growth factors to form mature dendritic cells and activated lymphocytes; and
c) administering a therapeutically effective amount of the composition to the patient.

30. The method of claim 28, wherein the administration is by systemic injection, intratumoral injection, or local injection into a lymph node draining the tumor.

31. The method of claim 28, wherein the tumor is melanoma, metastatic melanoma, basal cell carcinoma, squamous cell carcinoma, Merkel cell carcinoma, breast cancer, colon cancer, rectal cancer, cervical cancer, oral cancer, Hodgkin's lymphoma, non-Hodgkin's lymphoma, sarcoma, cancer of the head and neck, esophageal cancer, bladder cancer, prostate cancer, or cancer of the peritoneal lining.

32. (canceled)

33. A tumor vaccine comprising a composition of claim 20, a tumor antigen, and an adjuvant.

34. The method of claim 2, wherein;
(i) the leukocytes are incubated in the supportive medium for about 36 to 84 hours; or
(ii) the leukocytes are incubated in the supportive medium for about 48-72 hours.

35. The method of claim 3, wherein:
(i) the leukocytes are incubated in the supportive medium for about 36 to 84 hours; or
(ii) the leukocytes are incubated in the supportive medium for about 48-72 hours.

36. The method of claim 2, wherein:
(i) the composition is enriched in mature dendritic cells;
(ii) the composition further comprises activated lymphocytes;
(iii) the composition further comprises T helper cells enriched in Th1 phenotype;
(iv) the composition is enriched in Th1 cytokines;
(v) the composition further comprises active macrophages enriched in the M1 phenotype; and/or
(vi) the composition is enriched in M1 cytokines.

37. The method of claim 3, wherein
(i) the composition is enriched in mature dendritic cells;
(ii) the composition further comprises activated lymphocytes;
(iii) the composition further comprises T helper cells enriched in Th1 phenotype;
(iv) the composition is enriched in Th1 cytokines;
(v) the composition further comprises active macrophages enriched in the M1 phenotype; and/or
(vi) the composition is enriched in M1 cytokines.
